(19) United States
(12) Patent Application Publication
(10) Pub. No.: US 2020/0082910 A1
(43) Pub. Date: Mar. 12, 2020

(54) Systems and Methods for Determining Effects of Genetic Variation of Splice Site Selection

(71) Applicant: Deep Genomics Incorporated, Ontario (CA)

(72) Inventors: Brendan FREY, Ontario (CA); Hannes BRETSCHNEIDER, Ontario (CA)

(21) Appl. No.: 16/571,335

(22) Filed: Sep. 16, 2019

Related U.S. Application Data
(63) Continuation of application No. PCT/CA2018/050317, filed on Mar. 16, 2018.
(60) Provisional application No. 62/473,158, filed on Mar. 17, 2017.

(51) Int. Cl. G16B 30/00; G16B 20/20; G06N 3/08; G06N 20/10; C12Q 1/6869

(52) U.S. Cl. CPC G16B 30/00; G16B 20/20; C12Q 1/6869; G06N 20/10; G06N 3/08

(57) ABSTRACT

The present disclosure provides a computer-implemented method for determining a set of preferences, comprising: for an unspliced sequence of the one or more unspliced sequences, identifying (i) an anchor splice site comprising a location in the unspliced sequence, and (ii) a plurality of candidate complementary splice sites (n) corresponding to the anchor splice site, wherein each of the plurality of candidate complementary splice sites comprises a location in the unspliced sequence. A splice site feature vector for each of the plurality of candidate complementary splice sites and the anchor splice site may be calculated. Each of the splice site feature vectors may comprise one or more features determined based at least in part on one or more nucleotides in the unspliced sequence. A set of preferences p₁, p₂, . . . , pₙ corresponding to each of the plurality of candidate complementary splice sites may be calculated and outputted using the splice site feature vectors.

### CROSS-REFERENCE

This application is a continuation application of International Application No. PCT/CA2018/050317, filed Mar. 16, 2018, which claims the benefit of U.S. Provisional Patent Application No. 62/473,158, filed Mar. 17, 2017, each of which is hereby incorporated by reference in its entirety.

### BACKGROUND

Splicing is a natural biological mechanism that occurs within human cells and is used to process the primary messenger ribonucleic acid (mRNA) molecule that is transcribed from a deoxyribonucleic acid (DNA) molecule, before the mRNA molecule is translated into protein. Splicing may involve removing one or more contiguous segments of mRNA between pairs of 5′ splice sites and 3′ splice sites. Understanding how genetic variation influences the selection of these splice sites during the splicing process may yield important insights toward understanding and treating disease and other gross phenotypes.

### SUMMARY

The recent availability of datasets profiling the selection of splice sites from across the genome and in different cell lines, tissues and disease states, has made it possible to use machine learning to build systems that can ascertain the effect of genetic variation on splice site selection. This disclosure generally relates to a competitive model of splice site selection.

In an aspect, the present disclosure provides a computer-implemented method for determining a set of preferences corresponding to a plurality of candidate complementary splice sites, comprising: (a) providing one or more unspliced sequences in computer memory; and (b) for an unspliced sequence of the one or more unspliced sequences, i. identifying an anchor splice site comprising a location in the unspliced sequence; ii. identifying a plurality of candidate complementary splice sites (n) corresponding to the anchor splice site, wherein each of the plurality of candidate complementary splice sites comprises a location in the unspliced sequence; iii. using a computer to extract a splice site feature vector for each of the plurality of candidate complementary splice sites and the anchor splice site, wherein each of the splice site feature vectors comprises one or more features determined based at least in part on one or more nucleotides in the unspliced sequence; iv. using the splice site feature vectors for the plurality of candidate complementary splice sites and the anchor splice site to calculate a set of preferences p₁, p₂, . . . , pₙ corresponding to each of the plurality of candidate complementary splice sites; and v. outputting the set of preferences p₁, p₂, . . . , pₙ corresponding to the plurality of candidate complementary splice sites corresponding to the anchor splice site; and repeating (b) for any other unspliced sequence of the one or more unspliced sequences.

In some embodiments, each of the anchor splice sites is a 5′ splice site, and each of the plurality of candidate complementary splice sites corresponding to each of the anchor splice sites is a 3′ splice site. In some embodiments, each of the anchor splice sites is a 3′ splice site, and each of the plurality of candidate complementary splice sites corresponding to each of the anchor splice sites is a 5′ splice site.

In some embodiments, the calculation of the set of preferences comprises: (a) for each of the plurality of candidate complementary splice sites, using a preference computation module and the splice site feature vectors for the plurality of candidate complementary splice sites and the anchor splice site to calculate an intermediate representation rᵢ for an ith candidate complementary splice site, wherein the intermediate representation comprises at least one numerical value; and (b) calculating, using a normalization computation module and the set of intermediate representations r₁, r₂, . . . , rₙ for the plurality of candidate complementary splice sites, the set of preferences p₁, p₂, . . . , pₙ corresponding to the plurality of candidate complementary splice sites.

In some embodiments, at least one of the one or more unspliced sequences is (i) derived from a human genome or a genetic aberration thereof, or (ii) obtained by sequencing deoxyribonucleic acid (DNA) or unspliced ribonucleic acid (RNA) of a bodily sample obtained from a subject. In some embodiments, the at least one of the one or more unspliced sequences is (1) obtained by sequencing the DNA or unspliced RNA to obtain at least one genomic sequence, and (2) introducing the genetic aberration into the at least one genomic sequence. In some embodiments, the genetic aberration comprises a single nucleotide variant (SNV) or an insertion or deletion (indel).

In some embodiments, at least one splice site feature vector comprises a feature determined based at least in part on one or more nucleotides in the unspliced sequence, wherein the at least one of the one or more nucleotides are located within about 20 nucleotides of the location in the unspliced sequence of the anchor splice site. In some embodiments, at least one splice site feature vector comprises a feature determined based at least in part on one or more nucleotides in the unspliced sequence, wherein the at least one of the one or more nucleotides is located within about 20 nucleotides of the location in the unspliced sequence of the complementary splice site.

In some embodiments, each splice site feature vector comprises one or more of: (a) a subsequence of the unspliced sequence encoded using a 1-of-4 binary vector for a nucleotide selected from adenine (A), thymine (T), cytosine (C), and guanine (G); (b) a subsequence of the unspliced sequence encoded using a 1-of-4 binary vector for a nucleotide selected from adenine (A), uracil (U), cytosine (C), and guanine (G); (c) one or more binary components; (d) one or more categorical components; (e) one or more integer components; and (f) one or more real-valued components. In some embodiments, the one or more binary components comprise the presence (value of 1) or absence (value of 0), or vice versa, of a consensus dinucleotide sequence in the splice site. In some embodiments, the one or more binary components comprise the presence (value of 1) or absence (value of 0), or vice versa, of a consensus dinucleotide sequence adjacent to the splice site. In some embodiments, the one or more integer components comprise a distance, in number of nucleotides in the unspliced sequence, from (1) the candidate complementary splice site to (2) the anchor splice site to which the candidate complementary splice site corresponds. In some embodiments, the one or more real-valued components comprise a sequence of real values corresponding to the unspliced sequence, wherein each real value of the sequence is indicative of a probability that a corresponding nucleotide in the unspliced sequence is paired in a ribonucleic acid (RNA) secondary structure.

In some embodiments, the method further comprises (c) identifying a maximally preferred candidate complementary splice site among the plurality of candidate complementary splice sites with a largest numerical value of intermediate representation rₘₐₓ among the set of intermediate representations r₁, r₂, . . . , rₙ; and (d) outputting the maximally preferred candidate complementary splice site corresponding to the rₘₐₓ. In some embodiments, the method further comprises, for at least one of the one or more unspliced sequences: (c) identifying a maximally preferred candidate complementary splice site among the plurality of candidate complementary splice sites with a largest value of preference pₘₐₓ among the set of preferences p₁, p₂, . . . , pₙ; and (d) outputting the maximally preferred candidate complementary splice site corresponding to the pₘₐₓ.

In some embodiments, the calculation of the set of preferences comprises: (a) providing one or more numerical parameters; and (b) calculating a multiplication product comprising at least one feature from at least one splice site feature vector and at least one parameter of the one or more numerical parameters. In some embodiments, the calculation of the set of preferences further comprises applying a machine learning algorithm, which machine learning algorithm comprises adjusting at least one of the one or more numerical parameters to decrease a loss function. In some embodiments, adjusting the at least one of the one or more numerical parameters comprises performing a gradient-based machine learning procedure. In some embodiments, the loss function comprises a negative cross entropy represented by −Σᵢ₌₁ⁿpᵢ log \hat{p}ᵢ. In some embodiments, the loss function comprises a squared error represented by ½Σᵢ₌₁ⁿ(pᵢ−\hat{p}ᵢ)².

In some embodiments, a sum of the set of preferences p₁, p₂, . . . , pₙ equals 1. In some embodiments, each preference pᵢ among the set of preferences p₁, p₂, . . . , pₙ is indicative of a probability of selection of an ith candidate complementary splice site among the plurality of candidate complementary splice sites.

In some embodiments, the intermediate representation for the ith candidate complementary splice site comprises a numerical value rᵢ, and wherein the normalization computation module calculates each preference pᵢ as

$${p}_{i} = \frac{exp ( {r}_{i} )}{exp ( {r}_{1} ) + exp ( {r}_{2} ) + … + exp ( {r}_{n} )} ,$$

wherein exp is an exponential function or a numerical approximation of an exponential function. In some embodiments, the normalization computation module calculates each preference pᵢ as

$${p}_{i} = \frac{relu ( {r}_{i} )}{relu ( {r}_{1} ) + relu ( {r}_{2} ) + … + relu ( {r}_{n} )} ,$$

wherein relu is a rectified linear function. In some embodiments, the normalization computation module calculates each preference pᵢ as

$${p}_{i} = \frac{m ( {r}_{i} )}{m ( {r}_{1} ) + m ( {r}_{2} ) + … + m ( {r}_{n} )} ,$$

wherein m( ) is a non-negative monotonic function. In some embodiments, the intermediate representation for the ith candidate complementary splice site comprises a single numerical value rᵢ, and the normalization computation module: calculates, for each of the plurality of candidate complementary splice sites, an average intermediate representation aᵢ as aᵢ=(r₁+r₂+ . . . +rₙ)/n for an ith candidate complementary splice site; and calculates, using the intermediate representations for the plurality of candidate complementary splice sites r₁, r₂, . . . , rₙ and the average intermediate representation, the set of preferences p₁, p₂, . . . , pₙ corresponding to the plurality of candidate complementary splice sites. In some embodiments, the normalization computation module comprises a recurrent neural network, which recurrent neural network computationally processes the set of intermediate representations r₁, r₂, . . . , rₙ for the plurality of candidate complementary splice sites and outputs the set of preferences p₁, p₂ . . . , pₙ corresponding to the plurality of candidate complementary splice sites.

In some embodiments, the set of candidate complementary splice sites comprises known alternative complementary splice sites. In some embodiments, the set of candidate complementary splice sites comprises putative alternative complementary splice sites. In some embodiments, a putative alternative complementary splice site among the set of candidate complementary splice sites comprises a location in the unspliced sequence directly preceded by an AG (adenine-guanine) motif. In some embodiments, a putative alternative complementary splice site among the set of candidate complementary splice sites is identified by applying an existing splice site scoring system to the unspliced sequence.

In some embodiments, the one or more unspliced sequences comprises (1) an unspliced reference sequence and (2) an unspliced variant sequence corresponding to the unspliced reference sequence. In some embodiments, the method further comprises determining an effect of a genetic variant by processing the set of preferences corresponding to the plurality of complementary splice sites in the unspliced reference sequence with the set of preferences corresponding to the plurality of complementary splice sites in the unspliced variant sequence. In some embodiments, a one-to-one correspondence exists between one or more of the plurality of candidate complementary splice sites in the unspliced reference sequence and one or more of the plurality of candidate complementary splice sites in the unspliced variant sequence, and wherein processing the set of preferences corresponding to the plurality of complementary splice sites in the unspliced reference sequence with the set of preferences corresponding to the plurality of complementary splice sites in the unspliced variant sequence comprises processing each of at least one preference in the set of preferences corresponding to the plurality of complementary splice sites in the unspliced reference sequence with the corresponding preference in the set of preferences corresponding to the plurality of complementary splice sites in the unspliced variant sequence which is in one-to-one correspondence. In some embodiments, the set of preferences is outputted to a database. In some embodiments, the set of preferences is outputted to an electronic display.

Another aspect provides a computer-implemented method for determining an effect of one or more genetic variants on a set of anchor splice sites and corresponding complementary splice sites, comprising: (a) identifying a set of anchor splice sites in a human genome; (b) identifying one or more genetic variants in the human genome, wherein each of the one or more genetic variants comprises one or more aberrant nucleotides in the human genome; (c) for each anchor splice site in the set of anchor splice sites: (i) identifying a plurality of candidate complementary splice sites (n) corresponding to the anchor splice site, wherein each of the plurality of candidate complementary splice sites comprises a location in the human genome, (ii) using a computer to extract a splice site feature vector for each of the plurality of candidate complementary splice sites and the anchor splice site, wherein each of the splice site feature vectors comprises one or more features determined based at least in part on one or more nucleotides in the human genome, (iii) using a first preference computation module and the splice site feature vectors for the plurality of candidate complementary splice sites and the anchor splice site to calculate a set of intermediate representations r₁, r₂, . . . , rₙ corresponding to the plurality of candidate complementary splice sites corresponding to the anchor splice site, wherein each intermediate representation comprises at least one numerical value, and (iv) identifying an anchor splice site as affected by a genetic variant among the one or more genetic variants, when at least one of the one or more aberrant nucleotides of the genetic variant coincides with at least one of the one or more nucleotides in the human genome used to determine the one or more features of the splice site feature vectors for the plurality of candidate complementary splice sites corresponding to the anchor splice site, thereby identifying a set of affected anchor splice sites comprising at least a portion of the set of anchor splice sites; (d) for each affected anchor splice site in the set of affected anchor splice sites: (i) using a computer to extract modified feature vectors that comprise the one or more genetic variants for (1) each of the plurality of candidate complementary splice sites corresponding to the affected anchor splice site and (2) the affected anchor splice site, and (ii) using a second preference computation module and the splice site feature vectors for the plurality of candidate complementary splice sites and the affected anchor splice site to calculate a set of modified intermediate representations \tilde{r}₁, \tilde{r}₂, . . . , \tilde{r}ₙ corresponding to the plurality of candidate complementary splice sites corresponding to the affected anchor splice site, wherein each modified intermediate representation comprises at least one numerical value; (e) outputting the sets of intermediate representations r₁, r₂, . . . , rₙ corresponding to the set of anchor splice sites and the sets of modified intermediate representations \tilde{r}₁, \tilde{r}₂, . . . , \tilde{r}ₙ corresponding to the set of affected anchor splice sites; and (f) determining the effect of the one or more genetic variants on the set of affected anchor splice sites by processing the sets of intermediate representations r₁, r₂, . . . , rₙ with the sets of modified intermediate representations \tilde{r}₁, \tilde{r}₂, . . . , \tilde{r}ₙ across the set of affected anchor splice sites.

In some embodiments, each of the set of anchor splice sites is a 5′ splice site, and each of the plurality of candidate complementary splice sites corresponding to each of the set of anchor splice sites is a 3′ splice site. In some embodiments, each of the set of anchor splice sites is a 3′ splice site, and each of the plurality of candidate complementary splice sites corresponding to each of the set of anchor splice sites is a 5′ splice site.

In some embodiments, the method further comprises: (d) for each affected anchor splice site in the set of affected anchor splice sites: (iii) using a normalization computation module and the set of modified intermediate representations r₁, r₂, . . . , rₙ for the plurality of candidate complementary splice sites to calculate a set of modified preferences \tilde{p}₁, \tilde{p}₂, . . . , \tilde{p}ₙ corresponding to the plurality of candidate complementary splice sites corresponding to the affected anchor splice site; (e) outputting the sets of preferences p₁, p₂, . . . pₙ and the sets of modified preferences \tilde{p}₁, \tilde{p}₂, . . . , \tilde{p}ₙ corresponding to the set of affected anchor splice sites; and (f) determining the effect of the one or more genetic variants on the set of affected anchor splice sites by processing the sets of preferences p₁, p₂, . . . , pₙ with the sets of modified preferences \tilde{p}₁, \tilde{p}₂, . . . , \tilde{p}ₙ across the set of affected anchor splice sites. In some embodiments, the method further comprises: (d) for each affected anchor splice site in the set of affected anchor splice sites: (iv) calculating a set of changes in preference Δp₁, Δp₂, . . . , Δpₙ corresponding to the plurality of candidate complementary splice sites corresponding to the affected anchor splice site; (e) outputting the sets of changes in preference Δp₁, Δp₂, . . . , Δpₙ corresponding to each affected anchor splice site in the set of affected anchor splice sites; and (f) determining an effect of the one or more genetic variants on the set of affected anchor splice sites by processing the sets of changes in preference Δp₁, Δp₂, . . . , Δpₙ across the set of affected anchor splice sites. In some embodiments, the method further comprises: (d) for each affected anchor splice site in the set of affected anchor splice sites: (v) calculating a total probability mass change ΔP between the set of preferences p₁, p₂, . . . , pₙ and the set of modified preferences \tilde{p}₁, \tilde{p}₂, . . . , \tilde{p}ₙ; (e) outputting the set of total probability mass changes ΔP for each affected anchor splice site in the set of affected anchor splice sites; and (f) determining an effect of the one or more genetic variants on the set of affected anchor splice sites by processing the sets of total probability mass changes ΔP across the set of affected anchor splice sites.

In some embodiments, the human genome is obtained by sequencing deoxyribonucleic acid (DNA) or unspliced ribonucleic acid (RNA) of a bodily sample obtained from a subject. In some embodiments, at least one splice site feature vector comprises a feature determined based at least in part on one or more nucleotides in the unspliced sequence, wherein at least one of the one or more nucleotides are located within about 20 nucleotides of the location in the unspliced sequence of the anchor splice site. In some embodiments, at least one splice site feature vector comprises a feature determined based at least in part on one or more nucleotides in the unspliced sequence, wherein at least one of the one or more nucleotides are located within about 20 nucleotides of the location in the unspliced sequence of the complementary splice site.

In some embodiments, each of the one or more genetic variants comprises a sequential combination of one or more members selected from the group consisting of: (i) a substitution at one or more nucleotide positions relative to a reference sequence; (ii) an insertion at one or more nucleotide positions relative to a reference sequence; and (iii) a deletion at one or more nucleotide positions relative to a reference sequence.

In some embodiments, each splice site feature vector comprises one or more of: (a) a subsequence encoded using a 1-of-4 binary vector for each nucleotide selected from adenine (A), thymine (T), cytosine (C), or guanine (G); (b) a subsequence encoded using a 1-of-4 binary vector for each nucleotide selected from adenine (A), uracil (U), cytosine (C), or guanine (G); (c) one or more binary components; (d) one or more categorical components; (e) one or more integer components; and (f) one or more real-valued components. In some embodiments, the one or more binary components comprise the presence (value of 1) or absence (value of 0), or vice versa, of a consensus dinucleotide sequence in the splice site. In some embodiments, the one or more binary components comprise the presence (value of 1) or absence (value of 0), or vice versa, of a consensus dinucleotide sequence adjacent to the splice site. In some embodiments, the one or more integer components comprise a distance, in number of nucleotides in the human genome, from (1) the candidate complementary splice site to (2) the anchor splice site to which the candidate complementary splice site corresponds. In some embodiments, the one or more real-valued components comprise a sequence of real values corresponding to the unspliced sequence, wherein each real value of the sequence is indicative of a probability that a corresponding nucleotide in the unspliced sequence is paired in a ribonucleic acid (RNA) secondary structure.

In some embodiments, the method further comprises: (g) identifying a maximally preferred candidate complementary splice site among the plurality of candidate complementary splice sites with a largest numerical value of intermediate representation rₘₐₓ among the set of intermediate representations r₁, r₂, . . . rₙ; and (h) outputting the maximally preferred candidate complementary splice site corresponding to the rₘₐₓ. In some embodiments, the method further comprises, for at least one of the one or more unspliced sequences: (g) identifying a maximally preferred candidate complementary splice site among the plurality of candidate complementary splice sites with a largest numerical value of preference pₘₐₓ among the set of preferences p₁, p₂, . . . , pₙ; and (h) outputting the maximally preferred candidate complementary splice site corresponding to the pₘₐₓ. In some embodiments, the method further comprises: (g) identifying a maximally preferred candidate complementary splice site among the plurality of candidate complementary splice sites with a largest numerical value of modified intermediate representation \tilde{r}ₘₐₓ among the set of modified intermediate representations \tilde{r}₁, \tilde{r}₂, . . . , \tilde{r}ₙ; and (h) outputting the maximally preferred candidate complementary splice site corresponding to the \tilde{r}ₘₐₓ. In some embodiments, the method further comprises: (g) identifying a maximally preferred candidate complementary splice site among the plurality of candidate complementary splice sites with a largest numerical value of modified preference \tilde{p}ₘₐₓ among the set of modified preferences \tilde{p}₁, \tilde{p}₂, . . . , \tilde{p}ₙ; and (h) outputting the maximally preferred candidate complementary splice site corresponding to the \tilde{p}ₘₐₓ.

In some embodiments, the calculation of the set of preferences comprises: (a) providing one or more numerical parameters; and (b) calculating a multiplication product comprising at least one feature from at least one splice site feature vector and at least one parameter of the one or more numerical parameters. In some embodiments, the calculation of the set of preferences further comprises applying a machine learning algorithm, which machine learning algorithm comprises adjusting at least one of the one or more numerical parameters to decrease a loss function. In some embodiments, adjusting the at least one of the one or more numerical parameters comprises performing a gradient-based machine learning procedure. In some embodiments, the loss function comprises a negative cross entropy represented by −Σᵢ₌₁ⁿpᵢ log \hat{p}ᵢ. In some embodiments, the loss function comprises a squared error represented by ½Σᵢ₌₁ⁿ(pᵢ−\hat{p}ᵢ)².

In some embodiments, a sum of the set of preferences p₁, p₂, . . . , pₙ equals 1. In some embodiments, each preference pᵢ among the set of preferences p₁, p₂, . . . , pₙ is indicative of a probability of selection of an ith candidate complementary splice site among the plurality of candidate complementary splice sites. In some embodiments, the intermediate representation for the ith candidate complementary splice site comprises a numerical value rᵢ, and the normalization computation module calculates each preference pᵢ as

$${p}_{i} = \frac{exp ( {r}_{i} )}{exp ( {r}_{1} ) + exp ( {r}_{2} ) + … + exp ( {r}_{n} )} ,$$

wherein exp is an exponential function or a numerical approximation of an exponential function. In some embodiments, the normalization computation module calculates each preference pᵢ as

$${p}_{i} = \frac{relu ( {r}_{i} )}{relu ( {r}_{1} ) + relu ( {r}_{2} ) + … + relu ( {r}_{n} )} ,$$

wherein relu is a rectified linear function. In some embodiments, the normalization computation module calculates each preference pᵢ as

$${p}_{i} = \frac{m ( {r}_{i} )}{m ( {r}_{1} ) + m ( {r}_{2} ) + … + m ( {r}_{n} )} ,$$

wherein m( ) is a non-negative monotonic function.

In some embodiments, the set of candidate complementary splice sites comprises known alternative complementary splice sites. In some embodiments, the set of candidate complementary splice sites comprises putative alternative complementary splice sites. In some embodiments, a putative alternative complementary splice site among the set of candidate complementary splice sites comprises a location in the human genome directly preceded by an AG (adenine-guanine) motif. In some embodiments, a putative alternative complementary splice site among the set of candidate complementary splice sites is identified by applying an existing splice site scoring system to at least a portion of the human genome.

Another aspect provides a system for determining a set of preferences corresponding to a plurality of candidate complementary splice sites corresponding to an anchor splice site, comprising: a database comprising a human genome; and one or more computer processors operatively coupled to the database, wherein the one or more computer processors are individually or collectively programmed to: (i) provide one or more unspliced sequences, and (ii) for an unspliced sequence of the one or more unspliced sequences, (a) identify an anchor splice site comprising a location in the unspliced sequence; (b) identify a plurality of candidate complementary splice sites (n) corresponding to the anchor splice site, wherein each of the plurality of candidate complementary splice sites comprises a location in the unspliced sequence; (c) extract a splice site feature vector for each of the plurality of candidate complementary splice sites and the anchor splice site, wherein each of the splice site feature vectors comprises one or more features determined based at least in part on one or more nucleotides in the unspliced sequence; (d) use the splice site feature vectors for the plurality of candidate complementary splice sites and the anchor splice site to calculate a set of preferences p₁, p₂, . . . , pₙ corresponding to each of the plurality of candidate complementary splice sites; and (e) output the set of preferences p₁, p₂, . . . , pₙ corresponding to the plurality of candidate complementary splice sites corresponding to the anchor splice site; and (iii) repeat (ii) for any other unspliced sequence of the one or more unspliced sequences.

In some embodiments, each of the anchor splice sites is a 5′ splice site, and each of the plurality of candidate complementary splice sites corresponding to each of the anchor splice sites is a 3′ splice site. In some embodiments, each of the anchor splice sites is a 3′ splice site, and each of the plurality of candidate complementary splice sites corresponding to each of the anchor splice sites is a 5′ splice site.

Another aspect provides a non-transitory computer-readable medium comprising machine-executable code that, upon execution by one or more computer processors, implements a method for determining a set of preferences corresponding to a plurality of candidate complementary splice sites, the method comprising: (a) providing one or more unspliced sequences; and (b) for an unspliced sequence of the one or more unspliced sequences, i. identifying an anchor splice site comprising a location in the unspliced sequence; ii. identifying a plurality of candidate complementary splice sites (n) corresponding to the anchor splice site, wherein each of the plurality of candidate complementary splice sites comprises a location in the unspliced sequence; iii. extracting a splice site feature vector for each of the plurality of candidate complementary splice sites and the anchor splice site, wherein each of the splice site feature vectors comprises one or more features determined based at least in part on one or more nucleotides in the unspliced sequence; iv. using the splice site feature vectors for the plurality of candidate complementary splice sites and the anchor splice site to calculate a set of preferences p₁, p₂, . . . , pₙ corresponding to each of the plurality of candidate complementary splice sites; and v. outputting the set of preferences p₁, p₂, . . . , pₙ corresponding to the plurality of candidate complementary splice sites corresponding to the anchor splice site; and (c) repeating (b) for any other unspliced sequence of the one or more unspliced sequences.

In some embodiments, each of the anchor splice sites is a 5′ splice site, and each of the plurality of candidate complementary splice sites corresponding to each of the anchor splice sites is a 3′ splice site. In some embodiments, each of the anchor splice sites is a 3′ splice site, and each of the plurality of candidate complementary splice sites corresponding to each of the anchor splice sites is a 5′ splice site.

Another aspect provides a system for determining an effect of one or more genetic variants on a set of anchor splice sites and corresponding complementary splice sites, comprising: a database comprising a human genome; and one or more computer processors operatively coupled to the database, wherein the one or more computer processors are individually or collectively programmed to: (a) identify a set of anchor splice sites in a human genome; (b) identify one or more genetic variants in the human genome, wherein each of the one or more genetic variants comprises one or more aberrant nucleotides in the human genome; (c) for each anchor splice site in the set of anchor splice sites: (i) identify a plurality of candidate complementary splice sites (n) corresponding to the anchor splice site, wherein each of the plurality of candidate complementary splice sites comprises a location in the human genome, (ii) extract a splice site feature vector for each of the plurality of candidate complementary splice sites and the anchor splice site, wherein each of the splice site feature vectors comprises one or more features determined based at least in part on one or more nucleotides in the human genome, (iii) using a first preference computation module and the splice site feature vectors for the plurality of candidate complementary splice sites and the anchor splice site to calculate a set of intermediate representations r₁, r₂, . . . , rₙ corresponding to the plurality of candidate complementary splice sites corresponding to the anchor splice site, wherein each intermediate representation comprises at least one numerical value, and (iv) identify an anchor splice site as affected by a genetic variant among the one or more genetic variants, when at least one of the one or more aberrant nucleotides of the genetic variant coincides with at least one of the one or more nucleotides in the human genome used to determine the one or more features of the splice site feature vectors for the plurality of candidate complementary splice sites corresponding to the anchor splice site, to identify a set of affected anchor splice sites comprising at least a portion of the set of anchor splice sites; (d) for each affected anchor splice site in the set of affected anchor splice sites: (i) extract modified feature vectors that comprise the one or more genetic variants for (1) each of the plurality of candidate complementary splice sites corresponding to the affected anchor splice site and (2) the affected anchor splice site, and (ii) using a second preference computation module and the splice site feature vectors for the plurality of candidate complementary splice sites and the affected anchor splice site to calculate a set of modified intermediate representations \tilde{r}₁, \tilde{r}₂, . . . , \tilde{r}ₙ corresponding to the plurality of candidate complementary splice sites corresponding to the affected anchor splice site, wherein each modified intermediate representation comprises at least one numerical value; (e) output the sets of intermediate representations r₁, r₂, . . . , rₙ corresponding to the set of anchor splice sites and the sets of modified intermediate representations \tilde{r}₁, \tilde{r}₂, . . . , \tilde{r}ₙ corresponding to the set of affected anchor splice sites; and (f) determine the effect of the one or more genetic variants on the set of affected anchor splice sites by processing the sets of intermediate representations r₁, r₂, . . . , rₙ with the sets of modified intermediate representations \tilde{r}₁, \tilde{r}₂, . . . , \tilde{r}ₙ across the set of affected anchor splice sites.

In some embodiments, each of the set of anchor splice sites is a 5′ splice site, and each of the plurality of candidate complementary splice sites corresponding to each of the set of anchor splice sites is a 3′ splice site. In some embodiments, each of the set of anchor splice sites is a 3′ splice site, and each of the plurality of candidate complementary splice sites corresponding to each of the set of anchor splice sites is a 5′ splice site.

Another aspect provides a non-transitory computer-readable medium comprising machine-executable code that, upon execution by one or more computer processors, implements a method for determining an effect of one or more genetic variants on a set of anchor splice sites and corresponding complementary splice sites, the method comprising: (a) identifying a set of anchor splice sites in a human genome; (b) identifying one or more genetic variants in the human genome, wherein each of the one or more genetic variants comprise one or more aberrant nucleotides in the human genome; (c) for each anchor splice site in the set of anchor splice sites: (i) identifying a plurality of candidate complementary splice sites (n) corresponding to the anchor splice site, wherein each of the plurality of candidate complementary splice sites comprises a location in the human genome, (ii) extracting a splice site feature vector for each of the plurality of candidate complementary splice sites and the anchor splice site, wherein each of the splice site feature vectors comprises one or more features determined based at least in part on one or more nucleotides in the human genome, (iii) using a first preference computation module and the splice site feature vectors for the plurality of candidate complementary splice sites and the anchor splice site to calculate a set of intermediate representations r₁, r₂, . . . , rₙ corresponding to the plurality of candidate complementary splice sites corresponding to the anchor splice site, wherein each intermediate representation comprises at least one numerical value, and (iv) identifying an anchor splice site as affected by a genetic variant among the one or more genetic variants, when at least one of the one or more aberrant nucleotides of the genetic variant coincides with at least one of the one or more nucleotides in the human genome used to determine the one or more features of the splice site feature vectors for the plurality of candidate complementary splice sites corresponding to the anchor splice site, thereby identifying a set of affected anchor splice sites comprising at least a portion of the set of anchor splice sites; (d) for each affected anchor splice site in the set of affected anchor splice sites: (i) extracting modified feature vectors that comprise the one or more genetic variants for (1) each of the plurality of candidate complementary splice sites corresponding to the affected anchor splice site and (2) the affected anchor splice site, and (ii) using a second preference computation module and the splice site feature vectors for the plurality of candidate complementary splice sites and the affected anchor splice site to calculate a set of modified intermediate representations \tilde{r}₁, \tilde{r}₂, . . . , \tilde{r}ₙ corresponding to the plurality of candidate complementary splice sites corresponding to the affected anchor splice site, wherein each modified intermediate representation comprises at least one numerical value; (e) outputting the sets of intermediate representations r₁, r₂, . . . , rₙ corresponding to the set of anchor splice sites and the sets of modified intermediate representations \tilde{r}₁, \tilde{r}₂, . . . , \tilde{r}ₙ corresponding to the set of affected anchor splice sites; and (f) determining the effect of the one or more genetic variants on the set of affected anchor splice sites by processing the sets of intermediate representations r₁, r₂, . . . , rₙ with the sets of modified intermediate representations \tilde{r}₁, \tilde{r}₂, . . . , \tilde{r}ₙ across the set of affected anchor splice sites.

In some embodiments, each of the set of anchor splice sites is a 5′ splice site, and each of the plurality of candidate complementary splice sites corresponding to each of the set of anchor splice sites is a 3′ splice site. In some embodiments, each of the set of anchor splice sites is a 3′ splice site, and each of the plurality of candidate complementary splice sites corresponding to each of the set of anchor splice sites is a 5′ splice site.

Another aspect provides a method for identifying one or more phenotypes in a subject, comprising: (a) sequencing unspliced ribonucleic acid (RNA) molecules or deoxyribonucleic acid (DNA) molecules from a bodily sample obtained from the subject to produce a plurality of sequence reads; (b) using one or more programmed computer processors to (i) identify one or more genetic variants in the plurality of sequence reads or one or more sequences derived from the plurality of sequence reads, (ii) identify an anchor splice site associated with the one or more genetic variants, and (iii) identify a set of candidate complementary splice sites corresponding to the anchor splice site; (c) determining a set of preferences corresponding to the set of candidate complementary splice sites; and (d) using the set of preferences corresponding to the set of candidate complementary splice sites to identify the one or more phenotypes in the subject at a likelihood of occurrence of at least about 90%.

In some embodiments, the anchor splice site is a 5′ splice site, and each of the set of candidate complementary splice sites is a 3′ splice site. In some embodiments, the anchor splice site is a 3′ splice site, and each of the set of candidate complementary splice sites is a 5′ splice site.

In some embodiments, the method further comprises using a machine learning algorithm to identify the set of candidate complementary splice sites. In some embodiments, the set of candidate complementary splice sites comprises one or more complementary splice sites. In some embodiments, the likelihood of occurrence is at least about 95%, about 96%, about 97%, about 98%, or about 99%. In some embodiments, the method further comprises subjecting the RNA molecules to reverse transcription to generate complementary DNA (cDNA) molecules, and sequencing the cDNA molecules to produce the plurality of sequence reads. In some embodiments, the RNA is messenger RNA (mRNA).

Another aspect provides a non-transitory computer-readable medium comprising machine-executable code that, upon execution by one or more computer processors, implements a method for identifying one or more phenotypes in a subject, the method comprising: (a) sequencing ribonucleic acid (RNA) molecules or deoxyribonucleic acid (DNA) molecules from a bodily sample obtained from the subject to produce a plurality of sequence reads; (b) identifying (i) one or more genetic variants in the plurality of sequence reads, (ii) an anchor splice site associated with the one or more genetic variants, and (iii) a set of candidate complementary splice sites corresponding to the anchor splice site; (c) determining a set of preferences of the set of candidate complementary splice sites; and (d) using the set of preferences of the set of candidate complementary splice sites to identify the one or more phenotypes in the subject at a likelihood of occurrence of at least about 90%. In some embodiments, the anchor splice site is a 5′ splice site, and each of the set of candidate complementary splice sites is a 3′ splice site. In some embodiments, the anchor splice site is a 3′ splice site, and each of the set of candidate complementary splice sites is a 5′ splice site.

Another aspect provides a library of probes that enrich for a set of complementary splice sites in a nucleic acid sample of a subject, which set of complementary splice sites is generated using a preference computation module and corresponds to one or more genetic variants in the nucleic acid sample, and wherein the set of complementary splice sites identifies one or more phenotypes in the subject at a likelihood of occurrence of at least about 90%. In some embodiments, the likelihood of occurrence is at least about 95%, about 96%, about 97%, about 98%, or about 99%. In some embodiments, the set of complementary splice sites comprises one or more complementary splice sites.

Another aspect provides a system for identifying changes in one or more phenotypes in a subject, the system comprising: a database comprising a plurality of sequence reads generated from ribonucleic acid (RNA) or deoxyribonucleic acid (DNA) molecules; and one or more computer processors operatively coupled to the database, wherein the one or more computer processors are individually or collectively programmed to: (a) identify (i) one or more genetic variants in the plurality of sequence reads, (ii) an anchor splice site associated with the one or more genetic variants, and (iii) a set of candidate complementary splice sites corresponding to the anchor splice site; (b) determine a set of preferences of the set of candidate complementary splice sites; and (c) use the set of preferences of the set of candidate complementary splice sites to identify the one or more phenotypes in the subject at a likelihood of occurrence of at least about 90%. The RNA molecules may be messenger RNA (mRNA) molecules. In some embodiments, the anchor splice site is a 5′ splice site, and each of the set of candidate complementary splice sites is a 3′ splice site. In some embodiments, the anchor splice site is a 3′ splice site, and each of the set of candidate complementary splice sites is a 5′ splice site.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates a block diagram of a method capable of determining effects of genetic variation on splice site selection comprising generating with machine learning a prediction model for splicing preferences.

FIG. 2 illustrates a block diagram of a method to determine a set of normalized preferences of a plurality of candidate 3′ splice sites corresponding to a 5′ splice site.

FIG. 3A illustrates a block diagram of a method to evaluate effects of genetic variants (e.g., on 5′ splice sites, canonical 3′ splice sites, and/or candidate 3′ splice sites).

FIG. 3B illustrates a block diagram of a method to evaluate effects of genetic variants (e.g., on 5′ splice sites, canonical 3′ splice sites, and/or candidate 3′ splice sites).

FIG. 4 shows a computer system that is programmed or otherwise configured to implement methods provided herein.

### DETAILED DESCRIPTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The term “splice site,” as used herein, generally refers to a site in a genome corresponding to an end of an intron that may be involved in a splicing procedure. A splice site may be a 5′ splice site (e.g., a 5′ end of an intron) or a 3′ splice site (e.g., a 3′ end of an intron). A given 5′ splice site may be associated with one or more candidate 3′ splice sites, each of which may be coupled to its corresponding 5′ splice site in a splicing operation.

The term “sample,” as used herein, generally refers to a biological sample. A sample may be a fluid or tissue sample. The sample may include nucleic acid molecules, such as deoxyribonucleic acid (DNA) molecules, ribonucleic acid (RNA) molecules, or both. The RNA molecules may be messenger RNA (mRNA) molecules. The sample may be a tissue sample. The sample may be a cellular sample, such as a sample comprising one or more cells. The sample may be plasma, serum or blood (e.g., whole blood sample). The sample may be a cell-free sample (e.g., cell-free DNA, or cfDNA).

Splicing is a natural biological mechanism that may occur within human cells. Splicing processes primary messenger ribonucleic acid (mRNA) that has been transcribed from deoxyribonucleic acid (DNA), before the mRNA is translated into a protein. Splicing involves removing one or more contiguous segments of mRNA and is directed, in part, by a spliceosome. The segments that are removed are often referred to as introns, but the spliceosome may remove segments that contain both introns and exons.

During the splicing process, a spliceosome may detect the 5′ end of the segment that is to be removed (i.e., the 5′ splice site) and loop out the segment up to the 3′ end of the segment (i.e., the 3′ splice site). The 5′ end of the segment is close to the 3′ end of the segment, the 5′ end and the 3′ end of the segment are cut, and the two free ends of the mRNA with the segment removed are connected. The 5′ end of the segment may be bound to the branch point, which is close to the 3′ end of the segment, and the segment, which is lasso-shaped and is called a lariat, may be carried away.

The detection of the 5′ splice site, the 3′ splice site, the branch point, and other sequence features may depend on patterns within an unspliced mRNA sequence and corresponding patterns within a DNA sequence. Genetic variation in a nucleic acid (e.g., mutations in a DNA sequence or an mRNA sequence) may disrupt these patterns and cause different features to be detected. For instance, a 3′ splice site (e.g., a 3′ end of an intron) typically may be demarked by a dinucleotide consensus sequence comprising two nucleotides, A and G (e.g., an “AG motif”). If one of the two nucleotides in a dinucleotide consensus sequence is mutated in a nucleic acid (e.g., an mRNA strand or a DNA strand), the spliceosome may not detect the normal 3′ splice site and instead may detect a different 3′ splice site that is farther toward the 3′ end of the mRNA strand. Hence, the effect of this genetic variation may cause a different (e.g., longer or shorter) segment to be removed during splicing. This effect may result in functional consequences, such as one or more phenotype changes leading to a disease or acting as contributing factors in a disease. Many other sequence patterns may play important roles in splice site selection, e.g., a guanine-uracil (“GU”) dinucleotide consensus sequence at a 5′ end of an intron (a “GU” motif).

Understanding how genetic variation influences the selection of splice sites may be important for understanding disease, as well as other gross phenotypes, such as potentially for aging, developing therapies that act on RNA or DNA, and developing companion diagnostics that indicate under which genetic circumstances a therapy will be effective. For example, phenotypes (or changes in phenotypes) in a subject may be identified by comparison of a first set of preferences for selection of splice sites in an unspliced reference sequence (e.g., from a reference sequence such as a human genome) to a second set of preferences for selection of splice sites in an unspliced variant sequence (e.g., obtained by or derived from sequencing nucleic acids of the subject). For example, changes in preferred splice sites determined by analyzing genomic sequence derived from the subject may be indicative of likelihood of occurrence of phenotype changes in the subject.

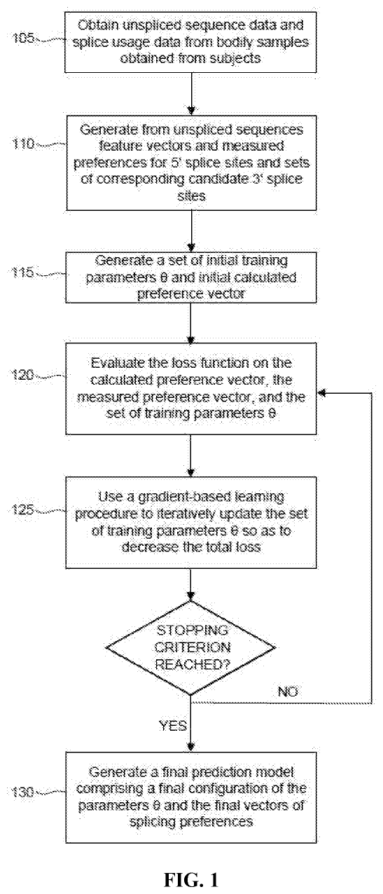

FIG. 1 illustrates a block diagram of a method capable of determining effects of genetic variation on splice site selection, comprising generating with machine learning a prediction model for splicing preferences. For a 5′ splice site and a corresponding set of candidate 3′ splice sites, the prediction model may calculate a feature vector y for the 5′ splice site and feature vectors x₁, . . . , xₙ for the n corresponding candidate 3′ splice sites, and may use these to calculate a set of preferences p₁, . . . , pₙ for the candidate 3′ splice sites. Different 5′ splice sites may have different numbers (n) of corresponding 3′ splice sites. This prediction model may comprise a first and/or a second preference computation module and a normalization computation module, as described elsewhere herein. A dataset of 5′ splice sites, corresponding candidate 3′ splice sites for each 5′ splice site, and/or the usage of the candidate 3′ splice sites may be used to adjust the parameters θ of the prediction model.

In operation 105, unspliced sequence data and splice site usage data may be obtained. For example, unspliced sequence data may be genomic sequences obtained or derived from a reference genome, by sequencing deoxyribonucleic acid (DNA) or unspliced ribonucleic acid (RNA) of one or more bodily samples obtained from one or more subjects, or by performing modifications (e.g., incorporating one or more genetic aberrations) of such genomic sequences. Such sequencing may be performed using next-generation sequencing (e.g., massively parallel sequencing or single molecule sequencing). The sequencing method can be massively parallel sequencing, e.g., simultaneously (or in rapid succession) sequencing of at least 100, 1000, 10,000, 100,000, 1 million, 10 million, 100 million, or 1 billion polynucleotide molecules. Examples of sequencing methods may include: next-generation sequencing, high-throughput sequencing, sequencing-by-synthesis, single-molecule sequencing, nanopore sequencing, pyrosequencing, semiconductor sequencing, sequencing-by-ligation, sequencing-by-hybridization, RNA-Seq (Illumina), Digital Gene Expression (Helicos), Single Molecule Sequencing by Synthesis (SMSS) (Helicos), massively parallel sequencing, Clonal Single Molecule Array (Solexa), shotgun sequencing, Maxam-Gilbert or Sanger sequencing, primer walking, sequencing using PacBio, SOLiD, Ion Torrent, or Oxford Nanopore platforms and any other sequencing methods known in the art.

A genetic aberration may be, for example, a single nucleotide variant (SNV) or an insertion or deletion (indel). Splice site usage data may be obtained using genome annotations, complementary DNA (cDNA) and expressed sequence tag (EST) libraries, or by sequencing spliced RNA of one or more bodily samples obtained from one or more subjects. A bodily sample may be derived from any organ, tissue or biological fluid. A bodily sample can comprise, for example, a bodily fluid or a solid tissue sample. An example of a solid tissue sample is a tumor sample, e.g., from a solid tumor biopsy. Bodily fluids may include, for example, blood, serum, plasma, tumor cells, saliva, urine, lymphatic fluid, prostatic fluid, seminal fluid, milk, sputum, stool, tears, and derivatives of these. Operation 105 may produce, for each of one or more unspliced sequences, a 5′ splice site and a set of candidate 3′ splice sites with corresponding measured preferences.

In operation 110, training cases may be obtained. Each training case may correspond to one 5′ splice site that is identified in the unspliced sequence data and a set of corresponding candidate 3′ splice sites that are identified in the unspliced sequence data. Each training case may comprise a feature vector y for the 5′ splice site, extracted from the unspliced sequence; feature vectors x₁, . . . , xₙ for the n corresponding candidate 3′ splice sites, extracted from the unspliced sequence; and measured preferences \hat{p}₁, . . . , \hat{p}ₙ for the candidate 3′ splice sites, extracted from the splice site usage data. In some embodiments, the measured preferences reflect the proportions of transcripts that map to the 5′ splice site and corresponding 3′ splice sites and sum to one, i.e., \hat{p}₁+\hat{p}₂+ . . . +\hat{p}ₙ=1. The set of measured preferences corresponding to the candidate 3′ splice sites may be denoted by \hat{p}=\hat{p}₁, . . . , \hat{p}ₙ, and the set of feature vectors x₁, . . . , xₙ corresponding to the candidate 3′ splice sites may be denoted by x=x₁, . . . , xₙ.

Using the 5′ splice site feature vector y, the 3′ splice site feature vectors x, and a set of parameters θ, a prediction model may calculate a set of preferences corresponding to the candidate 3′ splice sites, p₁, . . . , pₙ. Denoting these by p, where p=p₁, . . . , pₙ, the calculation performed by the prediction model may be denoted as p←f(x, y, θ).

In some embodiments, the feature vector for the ith candidate 3′ splice site, xᵢ, encodes the unspliced RNA sequence of length m centered on the 3′ splice site. The nucleotides adenine (A), cytosine (C), guanine (G), and uracil (U) may be encoded as (1,0,0,0), (0,1,0,0), (0,0,1,0) and (0,0,0,1), respectively, and the encodings of them nucleotides may be appended to form a binary sequence of length 4m. For example, for an unspliced RNA sequence GCAGCU3′GUUUCG, where 3′ indicates the 3′ splice site, and a window of size m=4, the feature vector may be expressed by (0, 1, 0, 0, 0, 0, 0, 1, 0, 0, 1, 0, 0, 0, 0, 1). The feature vector for the anchor (e.g., 5′) splice site may be computed in a similar manner. The prediction model may calculate the preferences p₁, . . . , pₙ by first calculating a set of corresponding intermediate representations r₁, . . . , rₙ, each of which may comprise a numerical value. The intermediate representation for the ith candidate 3′ splice site may be calculated using the following linear summation:

rᵢ←θ₁y₁+θ₂y₂+ . . . +θ₄ₘy₄ₘ+θ₄ₘ₊₁x_{i,1}+ . . . +θ₈ₘx_{i,4m}.

where the subscripts 1, 2, . . . , 4m index the elements of the binary sequences of length 4m, and each intermediate representation may comprise a sum of 8m terms. The intermediate representations may then be used to calculate the preferences as follows:

$${p}_{i} ← \frac{exp ( {r}_{i} )}{exp ( {r}_{1} ) + exp ( {r}_{2} ) + … + exp ( {r}_{n} )} , for i = 1 , … , n .$$

In another embodiment, the length of the 5′ splice site feature vector is different from the lengths of the 3′ splice site feature vectors. The feature vectors may encode other features, such as the presence of certain patterns such as a contiguous GU (e.g., guanine-uracil bases) at the 5′ splice site and a contiguous AG (e.g., adenine-guanine bases) at a the 3′ splice site; a numerical representation of RNA secondary structure; and a numerical encoding of nucleosome positioning. The intermediate representation for each 3′ splice site may comprise a single numerical value or a vector of numerical values, and may be calculated using a linear summation as shown above, a multilayer neural network comprised of multiple layers of computations with nonlinearities, a recurrent neural network, or one of many other types of machine learning systems. The intermediate representations for the 3′ splice sites may be combined using different computational approaches, such as those described elsewhere herein, to calculate the preferences.

In operation 115, a set of initial training parameters θ may be generated, e.g., by using preset values, by using a random number generator, or by setting them using additional data. A goal of training may be to adjust the parameters θ so that p and \hat{p} are close for every training case. Denoting the index of the training case by j, the 5′ splice site feature vector, the 3′ splice site feature vectors, the preferences corresponding to the candidate 3′ splice sites calculated by the prediction model, and the measured preferences corresponding to the candidate 3′ splice sites may be denoted respectively by: y^{j}, x^{j}, p^{j}, \hat{p}^{j}. These feature vectors and calculated preferences may be initialized, e.g., by setting all initial values to 0 or 1.

In operation 120, a loss function L(p^{j}, \hat{p}^{j}, θ) may be evaluated for the calculated preferences and the measured preferences, for the current set of training parameters θ. This loss function may depend on the parameters because the calculation of the preferences depends on the parameters, as described above.

Examples of suitable loss functions include a negative cross entropy loss function, represented by:

L=−Σᵢ₌₁ⁿpᵢ log \hat{p}ᵢ

or a squared error loss function, represented by:

L=½Σᵢ₌₁ⁿ(pᵢ−\hat{p}ᵢ)²;

alternatively, other loss functions may also be suitable.

In operation 125, a gradient-based machine learning procedure may be used to iteratively update the set of training parameters θ so as to decrease the total loss: L=L(p₁, \hat{p}₁, θ)+L(p₂, \hat{p}₂, θ)+ . . . +L(p_{T}, \hat{p}_{T}, θ), wherein T is the number of training cases. This operation 125 may be iterated until a stopping criterion is satisfied. Examples of stopping criteria are that a pre-determined number of iterations have been performed, a decrease in the total loss from one iteration to the next is below a pre-determined threshold, or the total loss evaluated on a held-out validation set (e.g., a subset of the training data set) increases instead of decreases. By considering a gradient of the total loss with respect to a single parameter

$$\frac{\partial L}{\partial {θ}_{j}} ,$$

a learning rate α, and iteratively generating small updates in a direction of the gradient:

$${θ}_{j}^{k + 1} ← {θ}_{j}^{k} + α \frac{\partial L}{\partial {θ}_{j}^{k}}$$

in its direction, the loss function can be minimized. For each iteration, a parameter update may be obtained by differentiating the selected loss function (to obtain a differential) and numerically evaluating the differential. The minimization of the loss function may result in more accurate predictions as training progresses iteratively. It will be appreciated that this gradient-based machine learning procedure may be combined with a variety of standard techniques, such as batch gradient descent, minibatch learning, stochastic gradient descent, learning with dropout, momentum-based learning methods, and others.

In operation 130, a final prediction model may be generated comprising a final configuration of the parameters θ, which may then be used to calculate the splicing preferences for any set of 5′ and 3′ splice site feature vectors. For training, it may be advantageous to alternate evaluation on randomized batches of training examples with parameter updates. As an example, a random set of training examples may be selected, the loss function may be evaluated based at least in part on this selected random set of training examples, gradients with respect to the model parameters may be computed, and the model parameters may be updated. This process may then be repeated with a different random set of training examples.

Since the same model may be applicable to examples with any number n of candidate 3′ splice sites, it may be advantageous to either only select training examples with the same number of candidate 3′ splice sites in one batch, or to select them such that the number of candidate 3′ splice sites in the same batch are not too dissimilar.

Whenever a single batch of training examples contains cases with different numbers of candidate 3′ splice sites (e.g., a “ragged batch”), one or more decoy inputs may need to be added to the cases with fewer candidate 3′ splice sites, thereby making all cases equal (e.g., having equal numbers of candidate 3′ splice sites) for computational reasons (e.g., a “balanced batch”), as well as mask out the preferences outputs corresponding to the decoy inputs.

The calculations made by the prediction model may be efficiently implemented on a graphics processing unit (GPU) for efficient training and for application at test time.

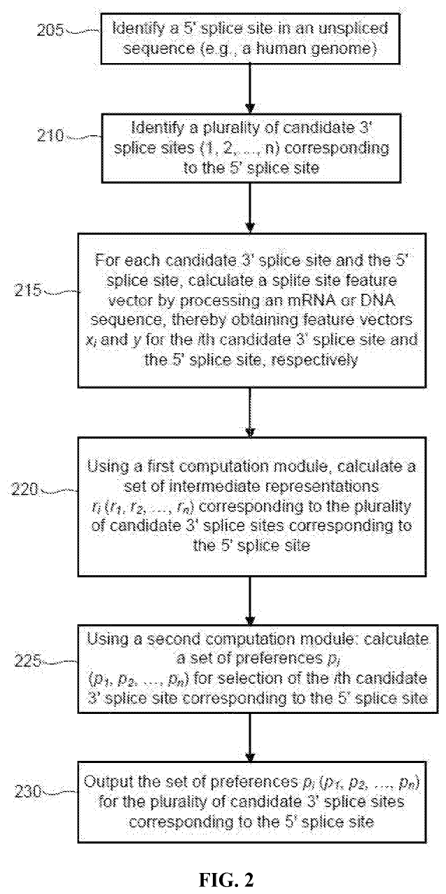

FIG. 2 illustrates a block diagram of a method 200 to determine a set of preferences of a plurality of candidate 3′ splice sites corresponding to a 5′ splice site.

In operation 205, a 5′ splice site may be identified in an unspliced sequence (e.g., a human genome). The 5′ splice site may comprise a contiguous segment of RNA (e.g., mRNA) or DNA. The 5′ splice site may correspond to a possible start of a spliced segment in the human genome. The human genome may be obtained by sequencing RNA (e.g., mRNA) or DNA of a bodily sample obtained from a subject. A bodily sample may be derived from any organ, tissue or biological fluid. A bodily sample can comprise, for example, a bodily fluid or a solid tissue sample. An example of a solid tissue sample is a tumor sample, e.g., from a solid tumor biopsy. Bodily fluids may include, for example, blood, serum, plasma, tumor cells, saliva, urine, lymphatic fluid, prostatic fluid, seminal fluid, milk, sputum, stool, tears, and derivatives of these.

In operation 210, a plurality of candidate 3′ splice sites (e.g., labeled 1, 2, . . . , n) may be identified in the unspliced sequence. Each candidate 3′ splice site among the plurality of candidate 3′ splice sites may comprise a contiguous segment of mRNA or DNA. Each candidate 3′ splice site among the plurality of candidate 3′ splice sites may correspond to a possible end of a spliced segment that corresponds to the 5′ splice site. This plurality of candidate 3′ splice sites may be referred to as a set of candidate 3′ splice sites (acceptor splice sites) conditional on one constitutive 5′ splice site (donor splice site). The set of candidate 3′ splice sites may comprise known (e.g., canonical) alternative 3′ splice sites. The set of candidate 3′ splice sites may comprise putative (e.g., non-canonical) alternative 3′ splice sites. For example, a putative alternative 3′ splice site among the set may comprise a nucleotide position preceded by an AG (adenine-guanine) motif within a predetermined window. As another example, a putative alternative 3′ splice site among the set may be identified using an existing splice site scoring system, such as MaxEntScan.

The systems and methods described herein can be trained to predict either (a) the utilization of a set of candidate 3′ splice sites (acceptor splice sites, or complementary splice sites) conditional on a constitutive 5′ splice site (donor splice site, or anchor splice site) or (b) the utilization of a set of candidate 5′ acceptor splice sites given a constitutive 3′ anchor splice site. While certain examples herein describe the former case (a), the latter case (b) may be constructed by readily substituting 5′ for 3′ and anchor for complementary (e.g., donor for acceptor), and vice versa. In some embodiments, each of the anchor splice sites is a 5′ splice site and each of the plurality of candidate complementary splice sites corresponding to each of the anchor splice sites is a 3′ splice site. Alternatively, each of the anchor splice sites may be a 3′ splice site and each of the plurality of candidate complementary splice sites corresponding to each of the anchor splice sites may be a 5′ splice site.

In operation 215, one or more splice site feature vectors may be calculated for each candidate 3′ splice site of the plurality of candidate 3′ splice sites and the corresponding 5′ splice site. The splice site feature vectors may be calculated by performing calculations on (e.g., processing) an mRNA sequence (or alternatively, a DNA sequence corresponding to the mRNA sequence) data. Performing operation 215 may result in extracting feature vectors xᵢ for the ith candidate 3′ splice site among the plurality of candidate 3′ splice sites and a feature vectory for the 5′ splice site.

Each feature vector (e.g., among the xᵢ or y feature vectors) may comprise a vector of one or more features determined (e.g., extracted) based at least in part on one or more nucleotide positions in the human genome. These features may be determined using other systems. A feature may be determined based at least in part on one or more nucleotides in the unspliced sequence. In some embodiments, the at least one of the one or more nucleotides are located within about 50, about 45, about 40, about 35, about 30, about 25, about 20, about 15, about 10, or about 5 nucleotides of the location in the unspliced sequence of the anchor (e.g., 5′) splice site. For example, a feature may comprise a raw sequence at a nucleotide position that may be encoded using a 1-of-4 binary vector for each nucleotide in a set of possible nucleotides for the sequence type (e.g., mRNA or DNA). For an mRNA sequence, a set of possible nucleotides may comprise adenine, “A”; uracil, “U”; cytosine, “C”; or guanine, “G.” For a DNA sequence, a set of possible nucleotides may comprise adenine, “A”; thymine, “T”; cytosine, “C”; or guanine, “G.” For instance, a 1-of-4 binary vector [0, 1, 0, 0]^{T} in an mRNA sequence may denote that a nucleotide located at a particular nucleotide position in the mRNA sequence is uracil, “U.” For instance, a 1-of-4 binary vector [0, 1, 0, 0]^{T} in a DNA sequence may denote that a nucleotide located at a particular nucleotide position in the DNA sequence is thymine, “T.”

A feature may comprise a binary component (value). For example, a feature may comprise a binary value indicating the presence (value of 1) or absence (value of 0), or vice versa, of a consensus dinucleotide sequence (e.g., an AG motif in a 3′ splice site). A feature may comprise categorical, integer, or real-valued components. For example, a feature may comprise an integer component such as a distance, in number of nucleotides, of a candidate 3′ splice site from the 5′ splice site to which the candidate 3′ splice site corresponds.

In operation 220, a preference computation module may be used to calculate a set of intermediate representations (r₁, r₂, . . . , rₙ) corresponding to the plurality (n) of candidate 3′ splice sites corresponding to a 5′ splice site. For each candidate 3′ splice site, a series of one or more structure computations may be performed on the feature vectors xᵢ and y to determine an intermediate representation rᵢ comprising one or more numerical values.

One of the values in the intermediate representations may indicate a preference of a candidate 3′ splice site relative to the other candidate 3′ splice sites in the plurality of candidate 3′ splice sites corresponding to the 5′ splice site. For instance, if intermediate representations are comprised of a single numerical value and if the first candidate 3′ splice site has a largest intermediate representation among the set of intermediate representations corresponding to the plurality of candidate 3′ splice sites corresponding to the 5′ splice site, then the first candidate 3′ splice site is the most likely to be selected (e.g., maximally preferred) as an actual 3′ splice site for the 5′ splice site by a spliceosome in a splicing process.

Once the intermediate representations for all of the candidate 3′ splice sites have been determined, r₁, r₂, . . . , rₙ they may be processed by a normalization computation module so that they may “compete” with one another to produce a set of preferences, p₁, p₂, . . . , pₙ. Thus, in operation 225, a normalization computation module may be used to calculate a set of preferences pᵢ (p₁, p₂, . . . , pₙ) for a selection of the ith candidate 3′ splice site among the plurality of candidate 3′ splice sites corresponding to the 5′ splice site. This may be performed using a normalization computation module denoted by p₁, p₂, . . . , pₙ←h(r₁, r₂, . . . , rₙ), where h is a pre-determined function on a set of one or more intermediate representation values.

For example, the normalization computation module may be operable to normalize the ith preference for a candidate 3′ splice site by using an exponential function for h, by assigning:

$${p}_{i} ← \frac{exp ( {r}_{i} )}{exp ( {r}_{1} ) + exp ( {r}_{2} ) + … + exp ( {r}_{n} )} ,$$

where exp( ) is an exponential function or a numerical approximation to an exponential function. As another example, the normalization computation module may be operable to normalize the ith preference for a candidate 3′ splice site by using a rectified linear function for h, by assigning:

$${p}_{i} ← \frac{relu ( {r}_{i} )}{relu ( {r}_{1} ) + relu ( {r}_{2} ) + … + relu ( {r}_{n} )} ,$$

where relu( ) is a rectified linear function, whose function output is equal to its input if the input is positive, or is equal to zero otherwise. The normalization computation module may be operable to normalize the ith preference for a candidate 3′ splice site by using another type of function for h. This function may be a monotonic function to preserve order of preferences between a set of intermediate representation values and a set of preference values.

Each preference pᵢ among the set of preferences (p₁, p₂, . . . , pₙ) may be indicative of a probability of selection of an ith candidate 3′ splice site among the plurality of candidate 3′ splice sites by a spliceosome in a splicing process with the 5′ splice site. As such, a sum of the set of preferences may equal one (e.g., p₁+p₂+ . . . +pₙ=1).

Operation 225 may further comprise identifying a maximally preferred candidate 3′ splice site among the plurality of candidate 3′ splice sites with a largest value of preference pₘₐₓ among the set of preferences (p₁, p₂, . . . , pₙ).

In operation 230, the set of preferences (p₁, p₂, . . . , pₙ) of the plurality of candidate 3′ splice sites corresponding to the 5′ splice site may be outputted by the computer-implemented method. For example, the set of preferences may be outputted to a database (e.g., by storing the set of preferences in the database). Alternatively or in combination, the set of preferences may be outputted to an electronic display (e.g., for display to a user).

If a maximally preferred candidate 3′ splice site corresponding to pₘₐₓ (a largest value of preference) was identified in operation 225, operation 230 may further comprise outputting the maximally preferred candidate 3′ splice site corresponding to pₘₐₓ.

It will be appreciated that an unspliced sequence, as described elsewhere herein, may be constructed by hand or by a computer by combining sequences from different sources, including spliced sequences. For example, a spliced mRNA molecule may be reverse transcribed into a complementary DNA (cDNA) molecule, and the resulting cDNA molecule may be sequenced to obtain a spliced sequence. This spliced sequence may be mapped to a genome (e.g., a human genome) by hand or by a computer, and the portions of the spliced sequence that were spliced out may be identified in the genome and inserted into the spliced sequence to form an unspliced sequence. It will be appreciated that there are different ways of assembling, by hand or by a computer, an unspliced sequence for the purposes described herein.

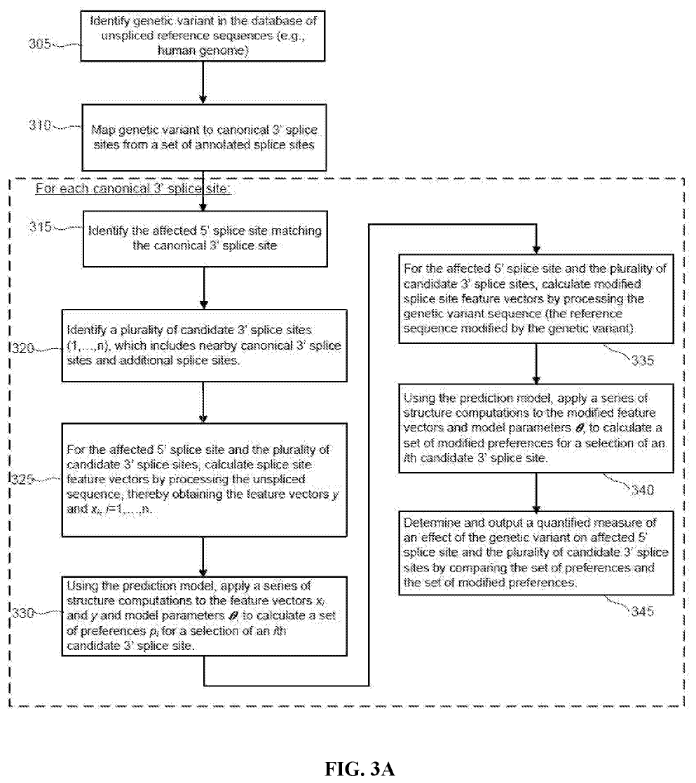

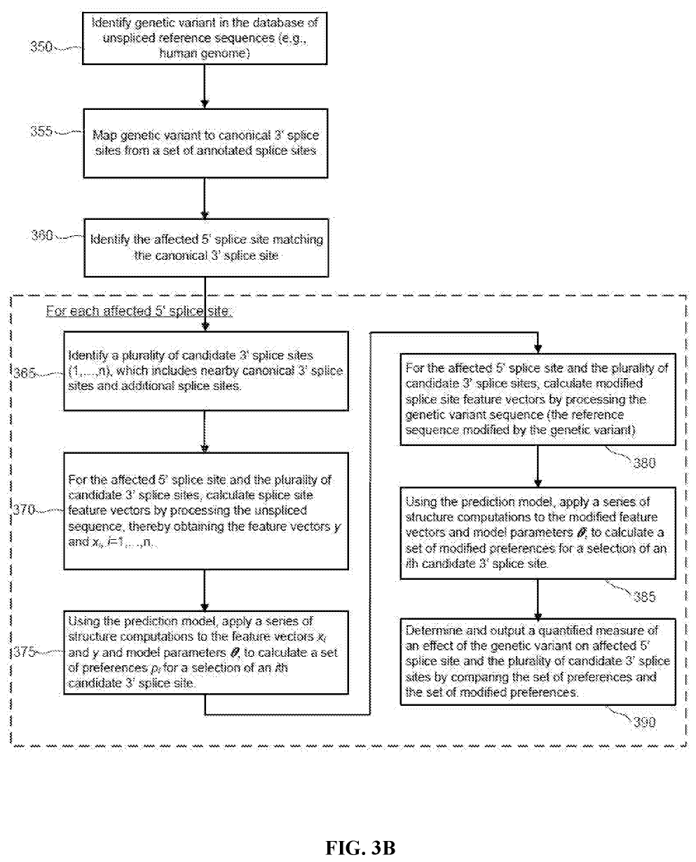

FIG. 3A illustrates a block diagram of a method to evaluate effects of genetic variants (e.g., on 5′ splice sites, canonical 3′ splice sites, and/or candidate 3′ splice sites). To evaluate a genetic variant, the variant may be specified with respect to an unspliced reference sequence, which may be derived from, e.g., the genome, DNA sequencing, sequencing an unspliced mRNA, or another approach. The variant may be specified by a sequential combination of one or more substitutions, insertions, and/or deletions with respect to the unspliced reference sequence. A substitution may be specified by a location in the reference sequence and the nucleotide (e.g., A, T, C, or G) that is substituted for the nucleotide at that location. An insertion may be specified by a location in the reference sequence and a nucleotide that is inserted right after the nucleotide at that location. A deletion may be specified by a location in the reference sequence at which a nucleotide has been removed from the sequence.

In some embodiments, the unspliced reference sequence is from the human genome, the unspliced reference sequence may be specified by a set of genomic coordinates, and the genetic variant may be specified by a series of substitutions, insertions, and deletions in the genome, as indicated using the set of genomic coordinates.

The system may maintain a database of unspliced sequences along with 5′ splice sites and corresponding canonical 3′ splice sites within the unspliced sequences. Canonical 3′ splice sites generally refer to 3′ splice sites that have been previously identified using, e.g., genome annotations, cDNA and EST data, RNA-Seq data, or another approach. The unspliced sequences may be represented as strings (e.g., a sequence) of letters (e.g., representing nucleotides), as substrings from a reference genome (e.g., a human genome), as pointers or genomic coordinates in a reference genome (e.g. a human genome), or another approach.

Referring to FIG. 3A, an embodiment is illustrated in which the human genome is used to represent the unspliced sequences. The operation 305 identifies one or more genetic variants in the database of unspliced reference sequences (e.g., a human genome). Each of the one or more genetic variants may comprise one or more aberrant nucleotide positions in the human genome. A genetic variant may be selected from the group consisting of: a substitution at one or more nucleotide positions relative to a reference sequence (e.g., a single nucleotide variant (SNV) or a single nucleotide polymorphism (SNP)), an insertion at one or more nucleotide positions relative to a reference sequence, and a deletion at one or more nucleotide positions relative to a reference sequence. An insertion or a deletion may be referred to as an indel. A reference sequence may comprise a portion or entirety of a human genome. For example, a reference sequence may comprise a portion or entirety of a human reference genome (e.g., GRCh38). Genetic variants may be identified using one or more databases of known variants. Genetic variants may be known to occur in a cohort of individuals with common characteristics, such as healthy subjects, subjects with a disease state or disorder state, subjects previously diagnosed with a disease state or disorder state, or subjects previously treated for a disease state or disorder state.

The operation 310 maps the genetic variant to canonical 3′ splice sites from a set of annotated splice sites. This mapping is used to identify canonical 3′ splice sites that may be affected by the genetic variant and may include 3′ splice sites wherein the adjacent nucleotides within a window of size W (e.g., in units of nucleotide locations) are altered by the genetic variant, or wherein the genetic variant alters nucleotides within a window of size W centered on neighboring upstream 5′ splice sites. It will be appreciated that canonical 3′ splice sites may be identified by other approaches. Each canonical 3′ splice site may comprise a contiguous segment of mRNA or DNA, or a location within a contiguous segment of mRNA or DNA. Each canonical 3′ splice site may correspond to a possible end of a spliced segment that corresponds to the 5′ splice site.

In operation 315, for each canonical 3′ splice site, the affected 5′ splice sites that match the canonical 3′ splice site are identified. This may be the first annotated 5′ splice site that is upstream of the canonical 3′ splice site, or the first plurality of annotated 5′ splice sites that are upstream of the canonical 3′ splice site. A 5′ splice site may comprise a contiguous segment of mRNA or DNA, or a location within a contiguous segment of mRNA or DNA. The 5′ splice site may correspond to a possible end of a spliced segment that corresponds to the canonical 3′ splice site.

In operation 320, for the affected 5′ splice site, a plurality of candidate 3′ splice sites are identified. A candidate 3′ splice site may comprise a contiguous segment of mRNA or DNA. A candidate 3′ splice site may correspond to a possible end of a spliced segment that corresponds to the 5′ splice site. This plurality of candidate 3′ splice sites may be referred to as a set of alternative 3′ splice sites (acceptor splice sites) conditional on one constitutive 5′ splice site (donor splice site). A set of candidate 3′ splice sites may comprise known (e.g., canonical) alternative 3′ splice sites. The plurality of candidate 3′ splice sites may include canonical 3′ splice sites that may be spliced to the affected 5′ splice site, as determined by examining annotations or cDNA/EST data or RNA-Seq data. The plurality of candidate 3′ splice sites may include additional putative 3′ splice sites that the genetic variant may introduce. For example, a segment of the unspliced reference sequence may comprise CCATGA, within which there are no canonical 3′ splice sites. If the genetic variant changes the T to a G, thereby resulting in a genetic variant sequence CCAGGA, then the pattern AG (adenine-guanine) within the genetic variant sequence is a possible putative 3′ splice site (CCAG3′GA), so it is included among the plurality of candidate 3′ splice sites. A putative 3′ splice site may comprise a nucleotide position preceded by an AG (adenine-guanine) motif within a predetermined window. As another example, a putative 3′ splice site may be identified using an existing splice site scoring system, such as MaxEntScan.

It may be acceptable for the identification of putative 3′ splice sites to have a higher false positive rate than is required by downstream applications, because the machine learning system described elsewhere herein may be capable of determining whether or not such identified putative 3′ splice sites are bona fide 3′ splice sites, thereby achieving a significantly lower false positive rate. In some embodiments, all nucleotide positions within some window downstream of the affected 5′ splice site are identified as putative 3′ splice sites and are included in the plurality of candidate 3′ splice sites, such as a window beginning at the affected 5′ splice site and ending at the canonical 3′ splice site.

Each candidate 3′ splice site in the plurality of candidate 3′ splice sites may comprise a contiguous segment of RNA (e.g., mRNA) or DNA, or a location within a contiguous segment of RNA or DNA.

In operation 325, for the affected 5′ splice site and the plurality of candidate 3′ splice sites, feature vectors y and xᵢ (for i=1, . . . , n) are calculated using the unspliced sequence, as described elsewhere herein. The unspliced sequence may be processed to extracted feature vectors. In operation 330, the prediction model is used to determine a set of preferences for the plurality of candidate 3′ splice sites, p₁, p₂, . . . , pₙ, as described elsewhere herein.

Referring again to FIG. 3A, in operation 335, the genetic variant sequence (the reference sequence modified by the genetic variant) is used to calculate modified feature vectors for the affected 5′ splice site and the plurality of 3′ splice sites, as described elsewhere herein. The modified feature vectors for the ith candidate 3′ splice site may be denoted by \tilde{x}ᵢ, and the modified feature vector for the affected 5′ splice site may be denoted by \tilde{y}. In operation 340, the prediction model is used to determine a set of modified preferences for the plurality of candidate 3′ splice sites, \tilde{p}₁, \tilde{p}₂, . . . , \tilde{p}ₙ, as described elsewhere herein.

In operation 345, the preferences for the plurality of candidate 3′ splice sites are processed with (e.g., compared to) the modified preferences for the plurality of candidate 3′ splice sites to determine a quantified measure of an effect of the genetic variant. Examples of possible methods of calculating this quantified measure are described elsewhere herein.

It will be appreciated that when identifying the affected 5′ splice site that matches each canonical 3′ splice site, multiple annotated 5′ splice sites may be identified and each of these may be used to determine the effect of the genetic variant on the corresponding plurality of candidate 3′ splice sites.

FIG. 3B illustrates a block diagram of a method to evaluate effects of genetic variants (e.g., on 5′ splice sites, canonical 3′ splice sites, and/or candidate 3′ splice sites). The operation 350 identifies the genetic variant in the database of unspliced reference sequences (e.g., a human genome). The operation 355 maps the genetic variant to canonical 3′ splice sites from a set of annotated splice sites. In operation 360, it will be appreciated that after the genetic variant is mapped to canonical 3′ splice sites, all corresponding affected 5′ splice sites may be identified. Then, each of the affected 5′ splice sites may be examined, e.g., in operations 365, 370, 375, 380, 385, and 390.

Operation 365 may be performed in a similar manner as operation 320, e.g., wherein for the affected 5′ splice site, a plurality of candidate 3′ splice sites are identified. Operation 370 may be performed in a similar manner as operation 325, e.g., wherein for the affected 5′ splice site and the plurality of candidate 3′ splice sites, feature vectors y and xᵢ (for i=1, . . . , n) are extracted using the unspliced sequence. Operation 375 may be performed in a similar manner as operation 330, e.g., wherein the prediction model is used to determine a set of preferences for the plurality of candidate 3′ splice sites, p₁, p₂, . . . , pₙ. Operation 380 may be performed in a similar manner as operation 335, e.g., wherein the genetic variant sequence (the reference sequence modified by the genetic variant) is used to extract modified feature vectors for the affected 5′ splice site and the plurality of 3′ splice sites. Operation 385 may be performed in a similar manner as operation 340, e.g., wherein the prediction model is used to determine a set of modified preferences for the plurality of candidate 3′ splice sites, \tilde{p}₁, \tilde{p}₂, . . . , \tilde{p}ₙ. Operation 390 may be performed in a similar manner as operation 345, e.g., wherein the preferences for the plurality of candidate 3′ splice sites are compared to the modified preferences for the plurality of candidate 3′ splice sites to determine a quantified measure of an effect of the genetic variant

It will be appreciated that as an alternative to comparing the set of preferences and the set of modified preferences when determining the quantified measure of the effect of the genetic variant, intermediate representations used in the calculations of the set of preferences and the set of modified preferences may be compared, as described elsewhere herein.

Each feature vector (e.g., among the xᵢ or y feature vectors) may comprise a vector of one or more features determined based at least in part on one or more nucleotide positions in the human genome. These features may be determined using other systems. For example, a feature may comprise a raw sequence at a nucleotide position that may be encoded using a 1-of-4 binary vector for each nucleotide in a set of possible nucleotides for the sequence type (e.g., mRNA or DNA). For an mRNA sequence, a set of possible nucleotides may comprise adenine, “A”; uracil, “U”; cytosine, “C”; or guanine, “G.” For a DNA sequence, a set of possible nucleotides may comprise adenine, “A”; thymine, “T”; cytosine, “C”; or guanine, “G.” For instance, a 1-of-4 binary vector [0, 1, 0, 0]^{T} in an mRNA sequence may denote that a nucleotide located at a particular nucleotide position in the mRNA sequence is uracil, “U.” For instance, a 1-of-4 binary vector [0, 1, 0, 0]^{T} in a DNA sequence may denote that a nucleotide located at a particular nucleotide position in the DNA sequence is thymine, “T.”

A feature may comprise a binary component (value). For example, a feature may comprise a binary value indicating the presence (value of 1) or absence (value of 0), or vice versa, of a consensus dinucleotide sequence (e.g., an AG motif in a 3′ splice site). A feature may comprise categorical, integer, or real-valued components. For example, a feature may comprise an integer component such as a distance, in number of nucleotides, of a candidate 3′ splice site from the 5′ splice site to which the candidate 3′ splice site corresponds.

For an affected 5′ splice site, a preference computation module may be used to calculate a set of intermediate representations (r₁, r₂, . . . , rₙ) for the selection of each candidate 3′ splice site (n) in the plurality of candidate 3′ splice sites corresponding to the affected 5′ splice site. For each candidate 3′ splice site, a series of one or more structured computations may be performed on the feature vectors xᵢ and y, and a numerical representation rᵢ (e.g., a real value, an integer, or a vector of numerical values) may be outputted.

The preference computation module may perform a series of structured computations that may be represented by rᵢ←f(xᵢ, y), where f denotes the series of one or more structure computations that are performed on the feature vectors xᵢ and y, and rᵢ is the intermediate representation for the selection of the ith candidate 3′ splice site in the plurality of candidate 3′ splice sites corresponding to the affected 5′ splice site. If the intermediate representation is a single numerical value, it may represent an un-normalized preference of a candidate 3′ splice site relative to the other candidate 3′ splice sites in the plurality of candidate 3′ splice sites corresponding to the 5′ splice site. For instance, if the first candidate 3′ splice site has a largest intermediate representation value among the set of un-normalized preference values corresponding to the plurality of candidate 3′ splice sites corresponding to the affected 5′ splice site, then the first candidate 3′ splice site is the most likely to be selected as an actual 3′ splice site for the affected 5′ splice site by a spliceosome in a splicing process.

In some embodiments, for each candidate splice site in the plurality of candidate 3′ splice sites, the calculation rᵢ←f(xᵢ, y) of the intermediate representation within the preference computation module is performed using a neural network, a deep neural network, a convolutional neural network, a recurrent neural network, a short-term long-term recurrent neural network, or another type of machine learning model. A convolutional or recurrent neural networks may process the feature vectors xᵢ and y separately, and the resulting hidden representation may be subsequently fed into another neural network. Alternatively, the feature vectors xᵢ and y may be concatenated to form one feature vector, which may be processed by a convolutional or a recurrent neural network, or some other type of neural network. It will be appreciated that the feature vectors may be assembled in various ways for processing within the preference computation module.

Once the intermediate representations for all of the candidate 3′ splice sites have been determined for each affected 5′ splice site in the set of affected 5′ splice sites, r₁, r₂, . . . , rₙ, they may “compete” with one another. This may be achieved using a normalization computation module that takes the intermediate representations r₁, r₂, . . . , rₙ as input, applies a series of structured computations, and outputs a set of preferences p₁, p₂, . . . , pₙ. The calculations performed by the normalization computation module may be denoted by p₁, p₂, . . . , pₙ←h(r₁, r₂, . . . , rₙ), where h is a pre-determined function of one or more intermediate representation values. This calculation may be performed using a neural network, a convolutional neural network, a recurrent neural network, a long-term short-term recurrent neural network, or another type of machine learning model. It will be appreciated that the intermediate representations may be assembled using various methods for processing within the preference computation module.

For example, for an affected 5′ splice site, the intermediate representation for each candidate 3′ splice site may be a single numerical value and the normalization computation module may be operable to normalize the ith intermediate representation for a candidate 3′ splice site corresponding to the affected 5′ splice site by using an exponential function for h, by assigning:

$${p}_{i} ← \frac{exp ( {r}_{i} )}{exp ( {r}_{1} ) + exp ( {r}_{2} ) + … + exp ( {r}_{n} )} ,$$

where exp( ) is an exponential function or a numerical approximation to an exponential function. As another example, for each affected 5′ splice site in the set of affected 5′ splice sites, the intermediate representation for each candidate 3′ splice site may be a single numerical value and the normalization computation module may be operable to normalize the ith intermediate representation for a candidate 3′ splice site corresponding to the 5′ splice site by using a rectified linear function for h, by assigning:

$${p}_{i} ← \frac{relu ( {r}_{i} )}{relu ( {r}_{1} ) + relu ( {r}_{2} ) + … + relu ( {r}_{n} )} ,$$

where relu( ) is a rectified linear function, whose function output is equal to its input if the input is positive, or is equal to zero otherwise. For each affected 5′ splice site in the set of affected 5′ splice sites, the normalization computation module may be operable to normalize the ith preference for a candidate 3′ splice site corresponding to the affected 5′ splice site by using another type of function for h. This function may be a monotonic function to preserve order of preferences between a set of intermediate representation values and a set of preference values.

Each preference pᵢ among the set of preferences (p₁, p₂, . . . , pₙ) may be indicative of a probability of selection of an ith candidate 3′ splice site among the plurality of candidate 3′ splice sites by a spliceosome in a splicing process with the 5′ splice site. As such, a sum of the set of normalized preferences may equal one (e.g., p₁+p₂+ . . . +pₙ=1).

Operation 330 and/or 375 may comprise, for each affected 5′ splice site in the set of affected 5′ splice sites, identifying a maximally preferred candidate 3′ splice site among the plurality of candidate 3′ splice sites corresponding to the affected 5′ splice site with a largest value of preference pₘₐₓ among the set of preferences (p₁, p₂, . . . , pₙ).

In some embodiments, for each affected 5′ splice site in a set of affected 5′ splice sites, a set of preferences of a plurality of candidate 3′ splice sites corresponding to the affected 5′ splice site may be obtained. An affected 5′ splice site may be identified as affected by a genetic variant among the one or more genetic variants, when one or more aberrant nucleotide positions of the genetic variant coincides with one or more nucleotide positions in the human genome used to determine features of splice site feature vectors for the plurality of candidate 3′ splice sites corresponding to the 5′ splice site. A nucleotide position in a human genome of a subject may comprise an aberrant nucleotide position when it differs from a nucleotide at the corresponding nucleotide position in a reference genome. A database may be maintained of 5′ splice sites and corresponding candidate 3′ splice sites. The database may also maintain the locations of the nucleotides that were used to derive the feature vectors for the candidate 3′ splice sites. For a genetic variant that is to be evaluated, a set of nucleotides that the genetic variant involves may be compared with a set of nucleotides that were used to derive the feature vectors for the candidate 3′ splice sites for every 5′ splice site. In this way, the affected 5′ splice sites may be identified. Thus, a set of affected 5′ splice sites may be identified, the set comprising at least a portion of the set of 5′ splice sites.

In operation 335 and/or 380, for each affected 5′ splice site in the set of affected 5′ splice sites, modified feature vectors may be calculated (e.g., extracted). The modified feature vectors may comprise the one or more genetic variants for (1) each of the plurality of candidate 3′ splice sites corresponding to the affected 5′ splice site and (2) the affected 5′ splice site. For each affected 5′ splice site, the modified feature vectors may be calculated using a modified sequence of the genetic variant (e.g., substitution, insertion, or deletion applied to the unspliced reference sequence, which may be derived from the human genome). The modified feature vectors for the ith candidate 3′ splice site may be denoted by \tilde{x}ᵢ, and the modified feature vector for the 5′ splice site may be denoted by \tilde{y}. A tilde symbol (“\tilde{ }”) may be used to denote a feature vector, an un-normalized preference, or a normalized preference that has been modified by a genetic variant.

In operation 340 and/or 385, for each affected 5′ splice site in the set of affected 5′ splice sites, a preference computation module may be used to calculate a set of modified intermediate representations \tilde{r}ᵢ(\tilde{r}₁, \tilde{r}₂, . . . , \tilde{r}ₙ) for the plurality of candidate 3′ splice sites corresponding to the affected 5′ splice site. This calculation may be represented by \tilde{r}ᵢ←f(\tilde{x}ᵢ,\tilde{y}), i=1, . . . , n, where f denotes the series of one or more structure computations that are performed on the modified feature vectors \tilde{x}ᵢ and \tilde{y}, and \tilde{r}ᵢ is the modified intermediate representation for the ith candidate 3′ splice site in the plurality of candidate 3′ splice sites corresponding to the affected 5′ splice site.

The modified intermediate representations \tilde{r}₁, \tilde{r}₂, . . . , \tilde{r}ₙ may be compared to the unmodified intermediate representations r₁, r₂, . . . , rₙ to determine the effect of the genetic variant.

Operation 340 and/or 385 may comprise, for each affected 5′ splice site in the set of affected 5′ splice sites, calculating, using a normalization computation module, a set of modified preferences \tilde{p}ᵢ (\tilde{p}₁, \tilde{p}₂, . . . , \tilde{p}ₙ) for the plurality of candidate 3′ splice sites corresponding to the affected 5′ splice site. This calculation may be denoted by \tilde{p}₁, \tilde{p}₂, . . . , \tilde{p}ₙ←h(\tilde{r}₁, \tilde{r}₂, . . . , \tilde{r}ₙ), where h is a pre-determined function on one or more modified intermediate representations.

For example, the intermediate representations may each comprise a single numerical value and normalization computation module may be operable to normalize the ith preference for a candidate 3′ splice site corresponding to an affected 5′ splice site in the set of affected 5′ splice sites by using an exponential function for h, by assigning:

$${\tilde{p}}_{i} ← \frac{exp ( {\tilde{r}}_{i} )}{exp ( {\tilde{r}}_{1} ) + exp ( {\tilde{r}}_{2} ) + … + exp ( {\tilde{r}}_{n} )} ,$$

where exp( ) is an exponential function or a numerical approximation to an exponential function. As another example, the normalization computation module may be operable to normalize the ith preference for a candidate 3′ splice site corresponding to an affected 5′ splice site in the set of affected 5′ splice sites by using a rectified linear function for h, by assigning:

$${\tilde{p}}_{i} ← \frac{relu ( {\tilde{r}}_{i} )}{relu ( {\tilde{r}}_{1} ) + relu ( {\tilde{r}}_{2} ) + … + relu ( {\tilde{r}}_{n} )} ,$$

where relu( ) is a rectified linear function, whose function output is equal to its input if the input is positive, or is equal to zero otherwise. The normalization computation module may be operable to normalize the ith preference for a candidate 3′ splice site corresponding to an affected 5′ splice site in the set of affected 5′ splice sites by using another type of function for h. This function may be a monotonic function to preserve order of preferences between a set intermediate representations and a set of preference values.

Each modified preference \tilde{p}ᵢ among the set of modified preferences (\tilde{p}₁, \tilde{p}₂, . . . , \tilde{p}ₙ) may be indicative of a probability of selection of an ith candidate 3′ splice site among the plurality of candidate 3′ splice sites by a spliceosome in a splicing process with the affected 5′ splice site. As such, a sum of the set of modified preferences may equal one (e.g., \tilde{p}₁+\tilde{p}₂+ . . . +\tilde{p}ₙ=1).

Operation 340 and/or 385 may comprise, for each affected 5′ splice site in the set of affected 5′ splice sites, identifying a maximally preferred candidate 3′ splice site among the plurality of candidate 3′ splice sites with a largest value of modified preference \tilde{p}ₘₐₓ among the set of preferences (\tilde{p}₁, \tilde{p}₂, . . . , \tilde{p}ₙ) corresponding to the affected 5′ splice site.

The effect of the genetic variant may be quantified by processing the preferences of the plurality of candidate 3′ splice sites with the modified preferences (e.g., by comparing the two). Based at least in part on this comparison, a quantitative measure may be generated and/or outputted. For example, if the maximally preferred candidate 3′ splice site in the modified and unmodified cases, pₘₐₓ and \tilde{p}ₘₐₓ, are different, a binary flag may be set to indicate a change.

If the intermediate representation is a single numerical value, operation 340 and/or 385 may comprise, for each affected 5′ splice site in the set of affected 5′ splice sites, identifying a maximally preferred candidate 3′ splice site among the plurality of candidate 3′ splice sites with a largest value of modified intermediate representation \tilde{r}ₘₐₓ among the set of intermediate representations (\tilde{r}₁, \tilde{r}₂, . . . , \tilde{r}ₙ) corresponding to the affected 5′ splice site.

Operation 340 and/or 385 may comprise, for each affected 5′ splice site in the set of affected 5′ splice sites, calculating a set of changes in preference Δpᵢ (Δp₁, Δp₂, . . . , Δpₙ) for the plurality of candidate 3′ splice sites corresponding to the affected 5′ splice site. Each change in preference may be represented by Δpᵢ=\tilde{p}ᵢ−pᵢ, Δpᵢ∈[−1, +1]. Alternatively, each change in preference may be computed using Δpᵢ=pᵢ log(pᵢ/\tilde{p}ᵢ), Δpᵢ∈[−1, +1]. It will be appreciated that the change in preference may be computed using various methods. The set of changes in preference may comprise a change in preference for a canonical 3′ splice site Δp_{c}, c∈{1, . . . , n}, which may be of particular interest and importance, since any deviation from the canonical 3′ splice site pattern may be indicative of pathogenicity. The canonical 3′ splice site may be determined by examining genome annotations, examining cDNA libraries, or by other approaches.

Operation 340 and/or 385 may comprise, for each affected 5′ splice site in the set of affected 5′ splice sites, calculating a total probability mass change ΔP between the set of preferences pᵢ and the set of modified preferences \tilde{p}ᵢ. The total probability mass change may be represented by: ΔP=½Σᵢ₌₁ⁿ|\tilde{p}ᵢ−pᵢ|, ΔP∈[0, 1]. In addition, for each affected 5′ splice site in the set of affected 5′ splice sites, a potentially cryptic splice site may be identified as a putative splice site (e.g., different from the canonical splice site) with a largest positive change in preference, represented as:

$$Δ {p}^{max} = {max}_{i \neq c} Δ {p}_{i} .$$

The preferences described above may be processed by another computation module that uses them to determine whether a specific disease is likely.

In operation 345 and/or 390, the sets of intermediate representations rᵢ and the sets of modified intermediate representations \tilde{r}ᵢ for the set of affected 5′ splice sites may be outputted by the computer-implemented method.

If, for each 5′ splice site in the set of affected 5′ splice sites, a maximally preferred candidate 3′ splice site corresponding to rₘₐₓ was identified, operation 345 and/or 390 may comprise outputting the maximally preferred candidate 3′ splice site corresponding to rₘₐₓ.

If, for each affected 5′ splice site in the set of affected 5′ splice sites, a maximally preferred candidate 3′ splice site corresponding to pₘₐₓ was identified, operation 345 and/or 390 may further comprise outputting the maximally preferred candidate 3′ splice site corresponding to pₘₐₓ.

If, for each affected 5′ splice site in the set of affected 5′ splice sites, a maximally preferred candidate 3′ splice site corresponding to \tilde{r}ₘₐₓ was identified, operation 345 and/or 390 may comprise outputting the maximally preferred candidate 3′ splice site corresponding to \tilde{r}ₘₐₓ.

If, for each affected 5′ splice site in the set of affected 5′ splice sites, a maximally preferred candidate 3′ splice site corresponding to \tilde{p}ₘₐₓ was identified, operation 345 and/or 390 may further comprise outputting the maximally preferred candidate 3′ splice site corresponding to \tilde{p}mₐₓ.

If, for each affected 5′ splice site in the set of affected 5′ splice sites, a set of changes in preference Δpᵢ (Δp₁, Δp₂, . . . , Δpₙ) was calculated, operation 345 and/or 390 may further comprise outputting the set of changes in preference Δpᵢ (Δp₁, Δp₂, . . . , Δpₙ).

If, for each affected 5′ splice site in the set of affected 5′ splice sites, a total probability mass change ΔP between the set of preferences pᵢ and the set of modified preferences \tilde{p}ᵢ was calculated, operation 345 and/or 390 may comprise outputting the total probability mass change ΔP.

In operation 345 and/or 390, an effect of the one or more genetic variants on the set of affected 5′ splice sites may be determined, by processing the sets of intermediate representations rᵢ (r₁, r₂, . . . , rₙ) with the sets of modified intermediate representations \tilde{r}ᵢ (\tilde{r}₁, \tilde{r}₂, . . . , \tilde{r}ₙ) across the set of affected 5′ splice sites (e.g., by comparing the two).

One or more phenotypes (or changes in one or more phenotypes) in a subject may be identified by sequencing ribonucleic acid (RNA) molecules or deoxyribonucleic acid (DNA) molecules from a bodily sample obtained from the subject to produce a plurality of sequence reads and identifying (e.g., using programmed computer processors) one or more genetic variants in the plurality of sequence reads or one or more sequences derived from the plurality of sequence reads. For example, the one or more sequences may be derived from the plurality of sequence reads by alignment to a reference sequence, assembly into contigs, collapsing, taking a subset, or a combination thereof. Next, a 5′ splice site associated with the one or more genetic variants may be identified, and a set of candidate 3′ splice sites corresponding to the 5′ splice site may be identified. A set of modified preferences of the set of candidate 3′ splice sites corresponding to the 5′ splice site may then be determined, and a set of preferences (e.g., normalized preferences) may also be determined using the reference sequence. These two sets of preferences may be processed (e.g., compared) to identify one or more phenotypes (or changes in one or more phenotypes) in the subject at a likelihood of occurrence (e.g., probability) of at least about 90%.

By determining a set of preferences of the set of candidate 3′ splice sites corresponding to a 5′ splice site associated with a genetic variant, the effect of the genetic variant may be determined as described elsewhere herein. This effect of the genetic variant may be used to identify one or more phenotypes (or changes in one or more phenotypes) in the subject at a likelihood of occurrence (e.g., probability) of at least about 50%, e.g., by performing correlation studies of cohorts of subjects with known genetic variants (e.g., DNA mutations) by comparing the changes in preferences to known changes in one or more phenotypes (e.g., diseases or disorders). The probability may be indicative of a likelihood that a subject with the genetic variant is exhibiting, will exhibit, or is expected to exhibit the change in one or more phenotypes. The probability may be at least about 55%, 60%, 65%, 70%, 75%, 80%, 85%, 90%, 91%, 92%, 93%, 94%, 95%, 96%, 97%, 98%, or 99%.

A machine learning algorithm may be used to identify the set of candidate 3′ splice sites corresponding to the affected 5′ splice site. The set of candidate 3′ splice sites may comprise one or more 3′ splice sites known to be associated with one or more spliced mRNA sequences.

The RNA molecules may be subjected to reverse transcription (e.g., RT) and/or reverse transcription polymerase chain reaction (e.g., RT-PCR) to generate complementary DNA (cDNA) molecules. The cDNA may then be sequenced to produce the plurality of sequence reads. The RNA molecules may be messenger RNA (mRNA).

A library of probes may be generated to enrich for a set of 3′ splice sites in a nucleic acid sample of a subject. The set of 3′ splice sites may be generated using a preference computation module, as described elsewhere herein, and may correspond to genetic variants in the nucleic acid sample. The set of 3′ splice sites may identify one or more phenotypes (or changes in one or more phenotypes) in the subject at a likelihood of occurrence (e.g., probability) of at least about 90%. The probability may be at least about 55%, 60%, 65%, 70%, 75%, 80%, 85%, 90%, 91%, 92%, 93%, 94%, 95%, 96%, 97%, 98%, or 99%. The set of candidate 3′ splice sites may comprise one or more 3′ splice sites known to be associated with one or more splicing events.

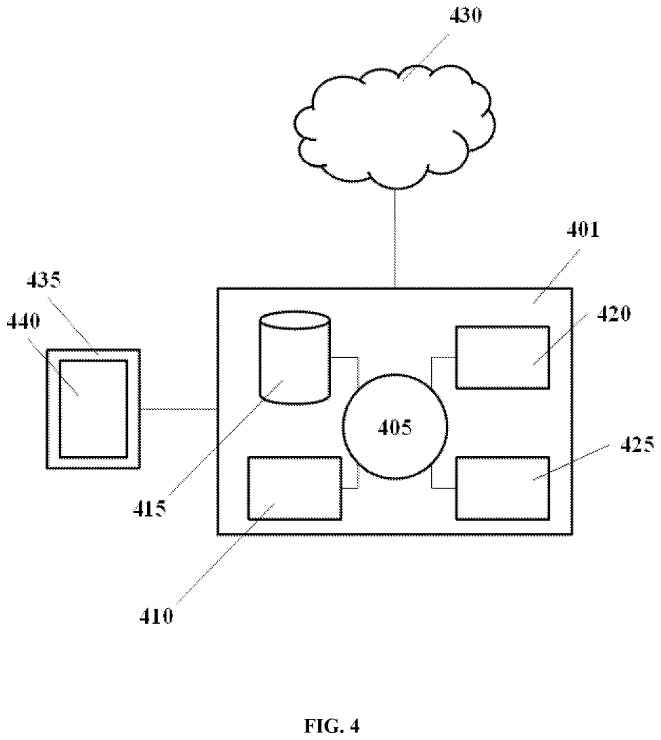

FIG. 4 shows a computer system that is programmed or otherwise configured to implement methods provided herein.

The present disclosure provides computer systems that are programmed to implement methods of the disclosure. FIG. 4 shows a computer system 401 that is programmed or otherwise configured to determine effect of a genetic variant on a set of 5′ splice sites. The computer system 401 can regulate various aspects of the preference computation module of the present disclosure, such as, for example, determining a set of preferences of a plurality of candidate 3′ splice sites corresponding to a 5′ splice site, implementing a preference computation module, and implementing a normalization computation module. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, an approach for user selection of a monotonic function and an output of a set of preferences to a user. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, determine a set of normalized preferences of a plurality of candidate 3′ splice sites corresponding to a 5′ splice site, implement a preference computation module, and implement a normalization computation module.

Although the description has been described with respect to particular embodiments thereof, these particular embodiments are merely illustrative, and not restrictive. Features illustrated in the examples may be applied to other examples and implementations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer-implemented method for determining a set of preferences corresponding to a plurality of candidate complementary splice sites, comprising:
(a) providing one or more unspliced sequences in computer memory; and
(b) for an unspliced sequence of the one or more unspliced sequences,
i. identifying an anchor splice site comprising a location in the unspliced sequence;
ii. identifying a plurality of candidate complementary splice sites (n) corresponding to the anchor splice site, wherein each of the plurality of candidate complementary splice sites comprises a location in the unspliced sequence;
iii. using a computer to extract a splice site feature vector for each of the plurality of candidate complementary splice sites and the anchor splice site, wherein each of the splice site feature vectors comprises one or more features determined based at least in part on one or more nucleotides in the unspliced sequence;
iv. using the splice site feature vectors for the plurality of candidate complementary splice sites and the anchor splice site to calculate a set of preferences p₁, p₂, . . . , pₙ corresponding to each of the plurality of candidate complementary splice sites; and
v. outputting the set of preferences p₁, p₂, . . . , pₙ corresponding to the plurality of candidate complementary splice sites corresponding to the anchor splice site; and
(c) repeating (b) for any other unspliced sequence of the one or more unspliced sequences.

2. The method of claim 1, wherein each of the anchor splice sites is a 5′ splice site, and wherein each of the plurality of candidate complementary splice sites corresponding to each of the anchor splice sites is a 3′ splice site.

3. The method of claim 1, wherein each of the anchor splice sites is a 3′ splice site, and wherein each of the plurality of candidate complementary splice sites corresponding to each of the anchor splice sites is a 5′ splice site.

4. The method of claim 1, wherein the calculation of the set of preferences comprises:
(a) for each of the plurality of candidate complementary splice sites, using a preference computation module and the splice site feature vectors for the plurality of candidate complementary splice sites and the anchor splice site to calculate an intermediate representation rᵢ for an ith candidate complementary splice site, wherein the intermediate representation comprises at least one numerical value; and
(b) calculating, using a normalization computation module and the set of intermediate representations r₁, r₂, . . . , rₙ for the plurality of candidate complementary splice sites, the set of preferences p₁, p₂, . . . , pₙ corresponding to the plurality of candidate complementary splice sites.

5. The method of claim 1, wherein at least one of the one or more unspliced sequences is (i) derived from a human genome or a genetic aberration thereof, or (ii) obtained by sequencing deoxyribonucleic acid (DNA) or unspliced ribonucleic acid (RNA) of a bodily sample obtained from a subject.

6. The method of claim 5, wherein the at least one of the one or more unspliced sequences is (1) obtained by sequencing the DNA or unspliced RNA to obtain at least one genomic sequence, and (2) introducing the genetic aberration into the at least one genomic sequence.

7. The method of claim 5, wherein the genetic aberration comprises a single nucleotide variant (SNV) or an insertion or deletion (indel).

8. The method of claim 1, wherein at least one splice site feature vector comprises a feature determined based at least in part on one or more nucleotides in the unspliced sequence, wherein the at least one of the one or more nucleotides is located within about 20 nucleotides of the location in the unspliced sequence of the anchor splice site or the complementary splice site.

9. (canceled)

10. The method of claim 1, wherein each splice site feature vector comprises one or more of:
(a) a subsequence of the unspliced sequence encoded using a 1-of-4 binary vector for a nucleotide selected from adenine (A), thymine (T), cytosine (C), and guanine (G);
(b) a subsequence of the unspliced sequence encoded using a 1-of-4 binary vector for a nucleotide selected from adenine (A), uracil (U), cytosine (C), and guanine (G);
(c) one or more binary components;
(d) one or more categorical components;
(e) one or more integer components; and
(f) one or more real-valued components.

11. The method of claim 10, wherein the one or more binary components comprise the presence (value of 1) or absence (value of 0), or vice versa, of a consensus dinucleotide sequence in the splice site or adjacent to the splice site.

12. (canceled)

13. The method of claim 10, wherein the one or more integer components comprise a distance, in number of nucleotides in the unspliced sequence, from (1) the candidate complementary splice site to (2) the anchor splice site to which the candidate complementary splice site corresponds.

14. The method of claim 10, wherein the one or more real-valued components comprise a sequence of real values corresponding to the unspliced sequence, wherein each real value of the sequence is indicative of a probability that a corresponding nucleotide in the unspliced sequence is paired in a ribonucleic acid (RNA) secondary structure.

15. (canceled)

16. The method of claim 1, further comprising, for at least one of the one or more unspliced sequences:
(c) identifying a maximally preferred candidate complementary splice site among the plurality of candidate complementary splice sites with a largest value of preference pₘₐₓ among the set of preferences p₁, p₂, . . . , pₙ; and
(d) outputting the maximally preferred candidate complementary splice site corresponding to the pₘₐₓ.

17. The method of claim 1, wherein the calculation of the set of preferences comprises:
(a) providing one or more numerical parameters; and
(b) calculating a multiplication product comprising at least one feature from at least one splice site feature vector and at least one parameter of the one or more numerical parameters.

18. The method of claim 17, wherein the calculation of the set of preferences further comprises applying a machine learning algorithm, which machine learning algorithm comprises adjusting at least one of the one or more numerical parameters to decrease a loss function.

19. The method of claim 18, wherein adjusting the at least one of the one or more numerical parameters comprises performing a gradient-based machine learning procedure.

20. The method of claim 18, wherein the loss function comprises a negative cross entropy represented by −Σᵢ₌₁ⁿpᵢ log \hat{p}ᵢ or a squared error represented by ½Σᵢ₌₁ⁿ(pᵢ−\hat{p}ᵢ)².

21. (canceled)

22. (canceled)

23. The method of claim 1, wherein each preference pᵢ among the set of preferences p₁, p₂, . . . , pₙ is indicative of a probability of selection of an ith candidate complementary splice site among the plurality of candidate complementary splice sites.

24. The method of claim 4, wherein the intermediate representation for the ith candidate complementary splice site comprises a numerical value rᵢ, and wherein the normalization computation module calculates each preference pᵢ as
$${p}_{i} = \frac{exp ( {r}_{i} )}{exp ( {r}_{1} ) + exp ( {r}_{2} ) + … + exp ( {r}_{n} )} ,$$
wherein exp is an exponential function or a numerical approximation of an exponential function, as pᵢ=relu(rᵢ)/relu(r₁)+relu(r₂)+ . . . +relu(rₙ), wherein relu is a rectified linear function, or as pᵢ=m(rᵢ)/m(r₁)+m(r₂)+ . . . +m(rₙ), wherein m( ) is a non-negative monotonic function.

25. (canceled)

26. (canceled)

27. (canceled)

28. The method of claim 4, wherein the normalization computation module comprises a recurrent neural network, which recurrent neural network computationally processes the set of intermediate representations r₁, r₂, . . . , rₙ for the plurality of candidate complementary splice sites and outputs the set of preferences p₁, p₂, . . . , pₙ corresponding to the plurality of candidate complementary splice sites.

29. The method of claim 1, wherein the set of candidate complementary splice sites comprises known alternative complementary splice sites or putative alternative complementary splice sites.

30. (canceled)

31. The method of claim 30, wherein a putative alternative complementary splice site among the set of candidate complementary splice sites comprises a location in the unspliced sequence directly preceded by an AG (adenine-guanine) motif.

32. The method of claim 30, wherein a putative alternative complementary splice site among the set of candidate complementary splice sites is identified by applying an existing splice site scoring system to the unspliced sequence.

33. The method of claim 1, wherein the one or more unspliced sequences comprise (1) an unspliced reference sequence and (2) an unspliced variant sequence corresponding to the unspliced reference sequence, and wherein the method further comprises determining an effect of a genetic variant by processing the set of preferences corresponding to the plurality of complementary splice sites in the unspliced reference sequence with the set of preferences corresponding to the plurality of complementary splice sites in the unspliced variant sequence.

34. (canceled)

35. The method of claim 33, wherein a one-to-one correspondence exists between one or more of the plurality of candidate complementary splice sites in the unspliced reference sequence and one or more of the plurality of candidate complementary splice sites in the unspliced variant sequence, and wherein processing the set of preferences corresponding to the plurality of complementary splice sites in the unspliced reference sequence with the set of preferences corresponding to the plurality of complementary splice sites in the unspliced variant sequence comprises processing each of at least one preference in the set of preferences corresponding to the plurality of complementary splice sites in the unspliced reference sequence with the corresponding preference in the set of preferences corresponding to the plurality of complementary splice sites in the unspliced variant sequence which is in one-to-one correspondence.

36.-70. (canceled)

71. A system for determining a set of preferences corresponding to a plurality of candidate complementary splice sites corresponding to an anchor splice site, comprising:
a database comprising a human genome; and
one or more computer processors operatively coupled to the database, wherein the one or more computer processors are individually or collectively programmed to:
(i) provide one or more unspliced sequences, and
(ii) for an unspliced sequence of the one or more unspliced sequences,
(a) identify an anchor splice site comprising a location in the unspliced sequence;
(b) identify a plurality of candidate complementary splice sites (n) corresponding to the anchor splice site, wherein each of the plurality of candidate complementary splice sites comprises a location in the unspliced sequence;
(c) extract a splice site feature vector for each of the plurality of candidate complementary splice sites and the anchor splice site, wherein each of the splice site feature vectors comprises one or more features determined based at least in part on one or more nucleotides in the unspliced sequence;
(d) use the splice site feature vectors for the plurality of candidate complementary splice sites and the anchor splice site to calculate a set of preferences p₁, p₂, . . . , pₙ corresponding to each of the plurality of candidate complementary splice sites; and
(e) output the set of preferences p₁, p₂, . . . , pₙ corresponding to the plurality of candidate complementary splice sites corresponding to the anchor splice site; and
(iii) repeat (ii) for any other unspliced sequence of the one or more unspliced sequences.

72.-99. (canceled)
